(19) United States
(12) Patent Application Publication
(10) Pub. No.: US 2014/0079660 A1
(43) Pub. Date: Mar. 20, 2014

(54) CLEANSING COMPOSITION FOR SKIN OR HAIR

(71) Applicant: KAO CORPORATION, Tokyo (JP)

(72) Inventor: Yasuhiro DOI, Kainan-shi (JP)

(73) Assignee: KAO CORPORATION, Tokyo (JP)

(21) Appl. No.: 13/727,330

(22) Filed: Dec. 26, 2012

(30) Foreign Application Priority Data
Sep. 20, 2012 (JP) 2012-207642

(51) Int. Cl. A61K 8/46; A61Q 5/12; A61Q 19/00; A61Q 5/02; A61Q 19/10

(52) U.S. Cl. CPC A61K 8/466; A61Q 5/02; A61Q 19/10; A61Q 19/00; A61Q 5/12
USPC 424/70.24; 562/30; 514/772; 510/127; 510/130

(57) ABSTRACT

The present invention provides a cleansing composition for skin or hair which can provide a good rinse feel, give good combability and softness from rinsing to drying when the cleansing composition is applied to hair, and give good moist feeling to skin when the cleansing composition is applied to skin.

The cleansing composition for skin or hair is a cleansing composition comprising an internal olefin sulfonate (A) having 12 or more and 24 or less carbon atoms.

### FIELD OF THE INVENTION

The present invention relates to a cleansing composition for skin or hair such as a shampoo and a body shampoo.

### BACKGROUND OF THE INVENTION

A cleansing agent is required to have a variety of functions such as emulsifying or cleaning the components of dirt and stains such as oil. Especially, unlike an industrial cleaner, a laundry cleaner, and a house cleaner, it is considered important that a cleansing agent used for skin or hair has not only detergency, excellent foaming performance and creamy foam quality for easy wash, but also a favorable rinsability and a good feel after rinsing and drying. Particularly in the case of hair, sliding property, good finger combability and softness of the hair during rinsing and after drying are desired, and in the case of skin, such an impression is desired that freshness after towel drying and a moist feeling after drying is imparted to the skin washed with a cleansing agent.

Under the foregoing circumstances, olefin sulfonate, which is one of the anionic surfactants, is generally obtained by sulfonating olefin through reactions with a gaseous sulfur trioxide-containing gas, followed by neutralization and then hydrolysis of the resulting sulfonic acid. Olefin sulfonate is used in various cleansing agents.

For example, JP-A-2003-81935 discloses a cleansing composition containing a specific internal olefin sulfonate for the purposes of increasing the solubilizing ability, penetrating ability, and interfacial tension reducing ability, and describes that when the above cleansing composition is used as a shampoo, it lathers well without friction, and achieves an improved feel. Also, U.S. Pat. No. 5,078,916 discloses a cleansing composition containing a specific internal olefin sulfonate for the purposes of improving detergency, and describes examples of application to shampoos and the like.

Meanwhile, JP-A-01-151510 discloses a cleansing composition containing an olefin sulfonate and a low viscous hydrophobic silicone oil such as octamethyltetrasiloxane and decamethylpentasiloxane to improve smoothness and silky touch of hair after drying.

### SUMMARY OF THE INVENTION

The present invention provides a cleansing composition for skin or hair, comprising an internal olefin sulfonate (A) having 12 or more and 24 or less carbon atoms (hereinbelow, may also be referred to as “the cleansing composition of the present invention”).

### DETAILED DESCRIPTION OF THE INVENTION

The present invention provides a cleansing composition for skin or hair which can provide a good rinse feel, give good combability and softness from rinsing to drying when the cleansing composition is applied to hair, and give good moist feeling to skin when the cleansing composition is applied to skin.

In light of the above, the present inventors carried out various studies. As a result, they found that a cleansing composition which can impart good combability from rinsing to drying and manageability after drying to hair, and a sufficient moist feeling also to skin after application, while exhibiting an excellent rinse feel as a cleansing agent for skin or a cleansing composition for hair can be obtained by using an internal olefin sulfonate having specific number of carbon atoms.

The cleansing composition of the present invention can not only bring about a good rinse feel, but also, when applied to hair, impart good combability and softness from rinsing to drying, while when applied to skin, impart a good moist feeling to skin.

Hereinbelow, the present invention will be described in detail.

The cleansing composition of the present invention contains an internal olefin sulfonate (A) having 12 or more and 24 or less carbon atoms.

The reason is not clear why the cleansing composition according to the present invention provides a good rinse feel, and gives good combability and softness from rinsing to drying when the cleansing composition is applied to hair, and a good moist feeling to skin when the cleansing composition is applied to skin. It is presumed that the internal olefin sulfonate having 12 or more and 24 or less carbon atoms has proper hydrophobicity, and therefore bubbles are easily broken in a diluted region by rinsing, and washed off, while part of the internal olefin sulfonate is adsorbed on skin or hair to give good combability and softness and to improve a moist feeling to skin.

<Internal Olefin Sulfonate (A)>

From the viewpoint of improving detergency, foam quality, and foamability, and a good rinse feel, and also, imparting good combability and softness to hair during rinsing and after drying and a moist feeling to skin, the cleansing composition of the present invention contains an internal olefin sulfonate having 12 or more and 24 or less carbon atoms (hereinbelow, may also be referred to as a component (A)).

In the present invention, an internal olefin sulfonate is an olefin sulfonate obtained by sulfonating an internal olefin (an olefin having a double bond inside the olefin chain) as the raw material, followed by neutralization and then hydrolysis. It should be noted that the above internal olefin may also contain a trace amount of so-called α-olefin, in which a double bond is present at the C-1 position of the carbon chain. That is, sulfonation of an internal olefin quantitatively produces β-sultone, some of which are converted into γ-sultone and olefin sulfonic acid, which are further converted into hydroxyalkane sulfonate and olefin sulfonate in the process of neutralization and hydrolysis (for example, J. Am. Oil Chem. Soc. 69, 39 (1992)). Here, the hydroxyl group of the hydroxyalkane sulfonate thus obtained is present inside the alkane chain, and the double bond of the olefin sulfonate is present inside the olefin chain. Also, the product thus obtained is mainly a mixture of the aforementioned substances, which may partially contain a trace amount of hydroxyalkane sulfonate having a hydroxyl group at the end of the carbon chain or olefin sulfonate having a double bond at the end of the carbon chain. In the present specification, each of these products and a mixture thereof are collectively referred to as internal olefin sulfonate (component (A)), and each of the product is individually referred to as internal olefin sulfonate. It should be noted that hydroxyalkane sulfonate is referred to as the hydroxy form of an internal olefin sulfonate (hereinbelow, may also be referred to as HAS), and olefin sulfonate is referred to as the olefin form of an internal olefin sulfonate (hereinbelow, may also be referred to as IOS).

From the viewpoint of improving a rinse feel, good combability after rinsing and manageability after drying, and imparting a moist feeling to skin, the number of carbon atoms in the internal olefin sulfonate of the component (A) is 12 or more, preferably 14 or more, and more preferably 16 or more. Also, from the viewpoint of softness of the hair during rinsing, manageability after drying, and a moist feeling on the skin, the number of carbon atoms in the internal olefin sulfonate of the component (A) is 24 or less, preferably 20 or less, and more preferably 18 or less. Also, from the viewpoint of improving a rinse feel and imparting good combability after rinsing and manageability after drying to hair, and a moist feeling to skin, the number of carbon atoms in the internal olefin sulfonate of the component (A) is 12 or more and 24 or less, preferably 14 or more and 20 or less, and more preferably 16 or more and 18 or less. These hydroxy form and olefin form containing various numbers of carbon atoms are derived from an internal olefin to be used as the raw material, and a hydroxy form and an olefin form containing different numbers of carbon atoms from those described above may also be contained.

From the viewpoint of improving detergency, foam quality, foamability, a rinse feel, and the feel after rinsing, the mass content ratio of an internal olefin sulfonate having 16 carbon atoms to an internal olefin sulfonate having 18 carbon atoms (internal olefin sulfonate having 16 carbon atoms/internal olefin sulfonate having 18 carbon atoms) in the component (A) or the cleansing composition is preferably from 50/50 to 99/1, more preferably from 60/40 to 95/5, more preferably from 70/30 to 90/10, more preferably from 75/25 to 90/10, and even more preferably from 75/25 to 85/15.

It is to be noted that the aforementioned mass ratio may be measured by a high-performance liquid chromatograph-mass spectrometer (hereinbelow, abbreviated as HPLC-MS). Specifically, an internal olefin sulfonate having 16 carbon atoms and an internal olefin sulfonate having 18 carbon atoms are separated from the component (A) or the produced cleansing composition by HPLC, each of which may then be identified by analysis with MS, and from the HPLC-MS peak area of each internal olefin sulfonate, the mass ratio between them may be obtained.

From the viewpoint of improving detergency, foam quality, foamability, a rinse feel, and the feel after rinsing, the total content of an internal olefin sulfonate having 16 carbon atoms and an internal olefin sulfonate having 18 carbon atoms in the component (A) is preferably 50% by mass or more, more preferably 60% by mass, more preferably 70% by mass, more preferably 80% by mass or more, more preferably 90% by mass or more, and even more preferably 95% by mass or more. It should be noted that the upper limit of the aforementioned total content is 100% by mass.

As is apparent from the aforementioned production method, the sulfonate group of the internal olefin sulfonate of the component (A) is present in the carbon chain of an internal olefin sulfonate, namely inside the olefin chain or alkane chain, and the component (A) may partially contain a trace amount of an internal olefin sulfonate having a sulfonate group at the end of the carbon chain. In the present invention, from the viewpoint of foamability, it is preferable that the content of an internal olefin sulfonate in which the sulfonate group is present at the C-2 position of the carbon chain is low, while the content of an internal olefin sulfonate in which the sulfonate group is present further inside is high in the component (A). It should be noted that when the component (A) contains an internal olefin sulfonate having 16 carbon atoms and an internal olefin sulfonate having 18 carbon atoms, it is more preferable that the content of an internal olefin sulfonate in which the sulfonate group is present at the C-2 position of the carbon chain is low, with respect to both of the above internal olefin sulfonates having 16 and 18 carbon atoms.

From the viewpoint of improving lathering property, foam quality, and a rinse feel as well as imparting good combability after rinsing and manageability after drying to hair, and a moist feeling to skin, the content of an internal olefin sulfonate in which the sulfonate group is present at the C-2 position in the component (A) is preferably 20% by mass or less, more preferably less than 20% by mass, more preferably 19% by mass or less, more preferably less than 18% by mass, and even more preferably 17.5% by mass or less. Also, from the viewpoint of reducing the production cost and improving productivity, the lower limit of the content of an internal olefin sulfonate in which the sulfonate group is present at the C-2 position in the component (A) is preferably 5% by mass or more, more preferably 6% by mass or more, more preferably 7% by mass or more, more preferably 8% by mass or more, and even more preferably 9% by mass or more. Further, from the viewpoint of improving lathering property, foam quality, and a rinse feel as well as imparting good combability after rinsing and manageability after drying to hair, and a moist feeling to skin, and also from the viewpoint of reducing the production cost and improving productivity, the content of an internal olefin sulfonate in which the sulfonate group is present at the C-2 position in the component (A) is preferably 5% by mass or more and 20% by mass or less, more preferably 6% by mass or more and less than 20% by mass, more preferably 7% by mass or more and 19% by mass or less, more preferably 8% by mass or more and less than 18% by mass, and even more preferably 9% by mass or more and 17.5% by mass or less.

Also, from the viewpoint of improving lathering property, foam quality, and a rinse feel as well as imparting good combability after rinsing and manageability after drying to hair, and a moist feeling to skin, the content of an olefin sulfonate in which the sulfonate group is present at the C-1 position of the olefin chain or alkane chain in the component (A) is preferably 3.0% by mass or less, more preferably 2.5% by mass or less, more preferably 2.0% by mass or less, more preferably 1.5% by mass or less, and even more preferably 1.0% by mass or less. From the viewpoint of reducing the production cost and improving productivity, the lower limit of the aforementioned content is preferably 0.01% by mass or more.

Here, the position at which the sulfonate group of olefin chain or alkane chain in the internal olefin sulfonate is present depends on the position of a double bond in the raw material internal olefin, and the content of an internal olefin sulfonate in which the sulfonate group is present at the C-2 position in the component (A) is roughly consistent with the content of the raw material internal olefin in which the double bond in the raw material internal olefin is present at the C-2 position. It should be noted that the content of an internal olefin sulfonate in which the sulfonate group is present at the C-2 position in the component (A) may be measured by a method such as nuclear magnetic resonance spectroscopy.

Further, from the viewpoint of improving lathering property, foam quality, and a rinse feel as well as imparting good combability after rinsing and manageability after drying to hair, and a moist feeling to skin, the content of an internal olefin sulfonate in which the sulfonate group is present further inside than the C-3 position of the olefin chain or alkane chain in the component (A) is preferably 70% by mass or more, more preferably 75% by mass or more, and even more preferably 80% by mass or more.

The internal olefin sulfonate is preferably a mixture of the hydroxy form and the olefin form. From the viewpoint of improving productivity and reducing impurities, the mass content ratio (hydroxy form/olefin form) of the hydroxy form of an internal olefin sulfonate to the olefin form of an internal olefin sulfonate in the component (A) or the cleansing composition is preferably from 50/50 to 100/0, more preferably from 60/40 to 100/0, more preferably from 70/30 to 100/0, more preferably from 75/25 to 100/0, and even more preferably from 75/25 to 95/5.

The mass content ratio of the hydroxy form of an internal olefin sulfonate to the olefin form of an internal olefin sulfonate in the component (A) or the cleansing composition may be obtained by separating the hydroxy form and the olefin form from the component (A) or the produced cleansing composition by HPLC and then measuring the separated substances by the method described in Examples.

From the viewpoint of environmental stability, low irritation, and the like, and also from the viewpoint of improving a rinse feel as well as imparting good combability to hair and a moist feeling to skin, the content of the aforementioned component (A) in the cleansing composition of the present invention is preferably 0.1% by mass or more, more preferably 1% by mass or more, more preferably 2% by mass or more, and even more preferably 5% by mass or more. Also, from the viewpoint of improving manageability of hair after drying, the content of the aforementioned component (A) in the cleansing composition of the present invention is preferably 80% by mass or less, more preferably 50% by mass or less, more preferably 30% by mass or less, and even more preferably 20% by mass or less. Also, from the viewpoint of improving a rinse feel as well as imparting good combability after rinsing and manageability after drying to hair, and a moist feeling to skin, the content of the aforementioned component (A) in the cleansing composition of the present invention is preferably 0.1% by mass or more and 80% by mass or less, more preferably 1% by mass or more and 50% by mass or less, more preferably 2% by mass or more and 30% by mass or less, and even more preferably 5% by mass or more and 20% by mass or less.

The internal olefin sulfonate (A) is obtainable by sulfonating a raw material internal olefin having 12 to 24 carbon atoms, followed by neutralization and hydrolysis. No particular limitation is imposed on the conditions of sulfonation, neutralization, and hydrolysis, and for example, the conditions described in U.S. Pat. Nos. 1,633,184 and 2,625,150, and Tenside Surf. Det. 31 (5) 299 (1994) may be referred to.

As mentioned above, in the present invention, an internal olefin refers to an olefin substantially having a double bond inside the olefin chain. From the viewpoint of the lathering property and rinse feel of the cleansing composition, combability of the hair after rinsing, and imparting a moist feeling to skin, the number of carbon atoms in the internal olefin is preferably from 12 to 24, more preferably from 12 to 20, more preferably from 12 to 18, more preferably from 14 to 18, and even more preferably from 16 to 18. An internal olefin to be used as the raw material may be used singly, or a combination of two or more thereof may be used.

When an internal olefin sulfonate is obtained by sulfonating the raw material internal olefin, followed by neutralization and then hydrolysis, the content of an internal olefin in which the double bond is present at the C-2 position in the raw material internal olefin is preferably 30% by mass or less, more preferably 25% by mass or less, more preferably 20% by mass or less, more preferably less than 20% by mass, more preferably 19% by mass or less, and even more preferably 18% by mass or less from the viewpoint of acquiring lathering property and a creamy foam quality for easy washing, improving a rinse feel, and achieving a good feel after rinsing. Also, from the viewpoint of reducing the production cost and improving productivity, the lower limit of the aforementioned ratio is preferably 5% by mass or more, more preferably 6% by mass or more, more preferably 7% by mass or more, more preferably 8% by mass or more, and even more preferably 9% by mass or more. Also, from the viewpoint of acquiring lathering property and a creamy foam quality for easy washing, improving a rinse feel, and achieving a good feel after rinsing, and also from the viewpoint of reducing the production cost and improving productivity, the total content of an internal olefin in which the double bond is present at the C-2 position in the raw material internal olefin is preferably 5% by mass or more and 30% by mass or less, more preferably 6% by mass or more and 25% by mass or less, more preferably 7% by mass or more and 20% by mass or less, more preferably 8% by mass or more and less than 20% by mass, more preferably 9% by mass or more and 19% by mass or less, and even more preferably 9% by mass or more and 18% by mass or less.

Also, from the viewpoint of improving lathering property, foam quality, and a rinse feel as well as imparting good combability after rinsing and manageability after drying to hair, and a moist feeling to skin, the content of an olefin in which the double bond is present at the C-1 position, namely a olefin, in the raw material internal olefin is preferably 3.0% by mass or less, more preferably 2.5% by mass or less, more preferably 2.0% by mass or less, more preferably 1.5% by mass or less, and even more preferably 1.0% by mass or less. From the viewpoint of reducing the production cost and improving productivity, the lower limit of the aforementioned content is preferably 0.01% by mass or more.

Further, from the viewpoint of improving lathering property, foam quality, and a rinse feel as well as imparting good combability after rinsing and manageability after drying to hair and a moist feeling to skin, the total content of an internal olefin in which the double bond is present further inside than the C-3 position in the raw material internal olefin is preferably 70% by mass or more, more preferably 75% by mass or more, and even more preferably 80% by mass or more.

The distribution of the double bond in the raw material internal olefin may be measured by, for example, a gas chromatograph mass spectrometer (hereinbelow, abbreviated as GC-MS). Specifically, components each having different carbon chain lengths and double bond positions are accurately separated by a gas chromatograph analyzer (hereinbelow, abbreviated as GC), and each component is then analyzed by a mass spectrometer (hereinbelow, abbreviated as MS) to identify the position of double bond, and from the resulting GC peak area, the fraction of each component may be found out.

The aforementioned sulfonation reaction may be carried out by reacting a sulfur trioxide gas with an internal olefin at a ratio of from 1.0 to 1.2 moles of sulfur trioxide per mole of the internal olefin. The reactions are preferably carried out at a reaction temperature of 20 to 40° C.

Neutralization is carried out by reacting from 1.0 to 1.5 times the molar amount of an alkali agent such as sodium hydroxide, ammonia, or 2-aminoethanol with the theoretical value of sulfonate group.

The hydrolysis reaction may be carried out at from 90 to 200° C. for from 30 minutes to three hours in the presence of water. These reactions may be successively carried out. Also, upon completion of the reactions, the products may be purified by extraction, washing, and the like.

Also, in the production of the internal olefin sulfonate (A), the raw material internal olefin in which the number of carbon atoms is distributed in from 12 to 24 may be subjected to sulfonation, neutralization, and hydrolysis, or the raw material internal olefin having a uniform number of carbon atoms may be subjected to sulfonation, neutralization, and hydrolysis. Also, a plurality of internal olefin sulfonates each having different numbers of carbon atoms may be produced in advance and then mixed, as needed.

The cleansing composition of the present invention may contain a surfactant (hereinbelow, may also be referred to as a component (B)) other than the aforementioned internal olefin sulfonate (A), so long as the effects of the present invention are not impaired.

As the surfactant other than the aforementioned component (A), any surfactant which is normally used in pharmaceutical products, quasi-drugs, cosmetics, toiletries, general merchandise, and the like may be used, and specific examples thereof include an anionic surfactant, a nonionic surfactant, an amphoteric surfactant, and a cationic surfactant other than the aforementioned component (A). From the viewpoint of improving the cleansing property, foamability, and foam quality, the surfactant other than the aforementioned component (A) is preferably a nonionic surfactant or an amphoteric surfactant other than the aforementioned (A).

From the viewpoint of improving the lathering property and cleansing property, the anionic surfactant other than the aforementioned component (A) is preferably a sulfuric acid ester salt, a sulfonic acid salt, a carboxylic acid salt, a phosphoric acid ester salt, and an amino acid salt. Specific examples thereof include a sulfuric acid ester salt such as alkyl sulfate, alkenyl sulfate, polyoxyalkylene alkyl ether sulfate, polyoxyalkylene alkenyl ether sulfate, and polyoxyalkylene alkyl phenyl ether sulfate; a sulfonic acid salt such as sulfosuccinic acid alkyl ester salt, polyoxyalkylene sulfosuccinic acid alkyl ester salt, alkane sulfonate, acyl isethionate and acyl methyl taurate; a higher fatty acid salt having from 8 to 16 carbon atoms; a phosphoric acid ester salt such as alkyl phosphate and polyoxyalkylene alkyl ether phosphate; and an amino acid salt such as acyl glutamate, an alanine derivative, a glycine derivative, and an arginine derivative.

Also, from the viewpoint of cleansing property, foamability, and foam quality, and from the viewpoint of imparting finger combability and manageability of hair after drying, and moist feeling to skin, the aforementioned anionic surfactant preferably has an alkyl group or alkenyl group having from 8 to 20 carbon atoms, and more preferably has an alkyl group or alkenyl group having from 10 to 16 carbon atoms.

Among such anionic surfactants, alkyl sulfate such as sodium lauryl sulfate, polyoxyethylene alkyl ether sulfate such as sodium polyoxyethylene lauryl ether sulfate, a higher fatty acid salt such as potassium laurate, a sulfosuccinic acid alkyl ester salt such as sodium polyoxyethylene lauryl ether sulfosuccinnate, acyl glutamate such as sodium N-acyl-L-glutamate, an acyl sarcosinine salt, acyl glycine salt, acyl isethionate, acyl methyl taurate, or alkyl phosphate is preferable, and sodium lauryl sulfate, ammonium polyoxyethylene (1) lauryl ether sulfate (ammonium laureth-1 sulfate), sodium polyoxyethylene (2) lauryl ether sulfate (sodium laureth-2 sulfate), potassium laurate, sodium polyoxyethylene lauryl ether (2) sulfosuccinate (sodium laureth-2 sulfosuccinate), or sodium cocoyl glutamate is more preferable.

From the viewpoint of improving cleansing property and the stability of the cleansing composition, examples of the aforementioned nonionic surfactant include a polyethylene glycol type such as polyoxyethylene sorbitan fatty acid ester, polyoxyethylene sorbitol fatty acid ester, polyoxyethylene glycerin fatty acid ester, polyoxyethylene fatty acid ester, polyoxyethylene alkyl ether, polyoxyethylene alkyl phenyl ether, or polyoxyalkylene (hydrogenated) castor oil, a polyhydric alcohol type such as sucrose fatty acid ester, polyglycerol alkyl ether, polyglycerol fatty acid ester, or alkyl glycoside, and fatty acid alkanolamide.

The nonionic surfactant preferably has an alkyl group or alkenyl group having from 8 to 20 carbon atoms as the hydrophobic moiety from the viewpoint of cleansing property of the cleansing composition according to the present invention and the volume and quality of foam during cleansing, and from the viewpoint of imparting finger combability and manageability of hair after drying treated with the cleansing composition.

Among such nonionic surfactants, alkyl glycoside having from 8 to 18 carbon atoms, preferably from 8 to 12 carbon atoms such as decyl glucoside, polyoxyalkylene alkyl ether such as polyoxyethylene lauryl ether, and fatty acid monoalkanolamide such as coconut oil fatty acid monoethanolamide are preferable, and decyl glucoside, polyoxyethylene (3) lauryl ether (laureth-3), polyoxyethylene (16) myristyl ether (ceteareth-16), polyoxypropylene (5) polyoxyethylene (20) cetyl ether, polyoxyethylene (9) lauryl ether, polyoxyethylene (7), alkyl ether having from 11 to 15 carbon atoms, coconut oil fatty acid monoethanolamide, or coconut oil fatty acid N-methyl monoethanolamide is more preferable.

From the viewpoint of improving stability of foam and feel during rinsing, examples of the aforementioned amphoteric surfactant include a betaine surfactant such as imidazoline betaine, alkyldimethylaminoacetate betaine, fatty acid amidopropyl betaine, or sulfobetaine, and an amine oxide surfactant such as alkyl dimethyl amine oxide.

Among such amphoteric surfactants, from the viewpoint of cleansing property of the cleansing composition according to the present invention and the volume and quality of foam during cleansing, and from the viewpoint of improving finger combability and manageability of hair after drying treated with the cleansing composition, imidazoline betaine, sulfobetaine, fatty acid amidopropyl betaine, and the like are preferable, and specifically, coconut oil fatty acid amidopropyl betaine, lauryl carbomethoxy methyl hydroxy imidazolium betaine, or lauryl hydroxy sulfobetaine is more preferable.

From the viewpoint of improving foam quality, softness of hair, and moist feeling of skin, examples of the aforementioned cationic surfactant include a mineral acid or organic acid salt of the tertiary amine represented by the following general formula (1) and a quaternary ammonium salt-type surfactant represented by the following general formula (2).

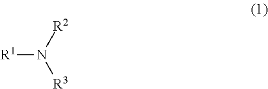

(wherein R¹ represents a linear or branched alkyl group or alkenyl group having from 6 to 28 carbon atoms which may be interrupted by an amide group, an ester group, or an ether group, and R² represents a linear or branched alkyl group, alkenyl group, or alkanol group having from 1 to 28 carbon atoms which may be interrupted by an amide group, an ester group, or an ether group, and R³ represents a linear or branched alkyl group or alkanol group having from 1 to 3 carbon atoms.)

In the general formula (1), from the viewpoint of improving finger combability and manageability of hair after drying treated with the cleansing composition according to the present invention, the number of carbon atoms in R¹ is preferably from 12 to 28, more preferably from 14 to 25, and even more preferably from 16 to 25. From a similar viewpoint, the number of carbon atoms in R² is preferably from 12 to 28, more preferably from 14 to 25, and even more preferably from 16 to 25, or R² is preferably a methyl group, an ethyl group, or a hydroxyethyl group. From a similar viewpoint, R³ is preferably a methyl group, an ethyl group, or a hydroxyethyl group.

No particular limitation is imposed on the mineral acid or organic acid which forms a salt with the tertiary amine represented by the general formula (1); from the viewpoint of dispersion stability of a surfactant, hydrogen halide, sulfuric acid, acetic acid, citric acid, lactic acid, glutamic acid, and alkyl sulfate having from 1 to 3 carbon atoms are preferable, and from the viewpoint of chemical stability, hydrogen halide is preferably hydrogen chloride.

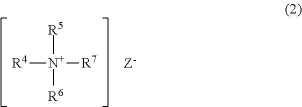

(wherein R⁴ represents a linear or branched alkyl group or alkenyl group having from 6 to 28 carbon atoms which may be interrupted by an amide group, an ester group, or an ether group, R⁵ represents a linear or branched alkyl group, alkenyl group, or alkanol group having from 1 to 28 carbon atoms which may be interrupted by an amide group, an ester group, or an ether group, R⁶ and R⁷ each represent a linear or branched alkyl group having from 1 to 3 carbon atoms, and Z− represents an anionic group, which is the counter ion of an ammonium salt.)

In the general formula (2), from the viewpoint of improving finger combability and manageability of hair after drying treated with the cleansing composition according to the present invention, a preferred embodiment of R⁴ is the same as a preferred embodiment of R¹ in the general formula (1). From a similar viewpoint, a preferred embodiment of R⁵ is the same as a preferred embodiment of R² in the general formula (1). Also, from a similar viewpoint, R⁶ and R⁷ are each preferably a methyl group or an ethyl group.

No particular limitation is imposed on Z⁻ as long as it is an anionic group. Specific examples thereof include an alkyl sulfate ion, a sulfate ion, a phosphate ion, alkyl carboxylate, and a halide ion. Among them, from the viewpoint of easiness of production and availability, a halide ion is preferable. Examples of the halide ion include a fluoride ion, a chloride ion, a bromide ion, and an iodide ion, and from the viewpoint of chemical stability, a chloride ion or a bromide ion is preferable, of which a chloride ion is more preferable.

Examples of the mineral acid or organic acid salt of the tertiary amine represented by the general formula (1) and the quaternary ammonium salt-type surfactant represented by the general formula (2) include mono long-chain alkyl trimethyl ammonium chloride, di long-chain alkyl dimethyl ammonium chloride, and a long-chain tertiary amine salt. Specific examples include mono long-chain alkyl trimethyl ammonium chloride such as stearyl trimethyl ammonium chloride, behenyl trimethyl ammonium chloride, cetyl trimethyl ammonium chloride, and stearoxy propyl trimethyl ammonium chloride; di long-chain alkyl dimethyl ammonium chloride such as distearyl dimethyl ammonium chloride and diisostearyl dimethyl ammonium chloride; mono long-chain dimethylamine such as stearyl dimethylamine, behenyl dimethylamine, octadecyloxypropyl dimethylamine, stearamidoethyl diethylamine, stearamidopropyl dimethylamine, and behenamidopropyl dimethylamine, a glutamic acid salt, a hydrochloric acid salt, a citric acid salt, or a lactic acid salt and the like of mono long-chain diethylamine. From the viewpoint of improving finger combability and manageability of hair after drying treated with the cleansing composition according to the present invention, behenyl trimethyl ammonium chloride, cetyl trimethyl ammonium chloride, stearoxy propyl trimethyl ammonium chloride, stearyl dimethylamine, stearamidopropyl dimethylamine, and behenamidopropyl dimethylamine are preferable.

From the viewpoint of improving combability during rinsing and softness of hair, and moist feeling of skin according to the cleansing composition, the content of the aforementioned component (B) in the cleansing composition of the present invention is preferably 50% by mass or less, more preferably 30% by mass or less, more preferably 20% by mass or less, more preferably 15% by mass or less, more preferably 10% by mass or less, more preferably 5% by mass or less, and even more preferably 1% by mass or less.

The mass content ratio of the component (A) to the component (B) [component (A)/component (B)] is preferably from 1000 to 0.01, more preferably from 100 to 0.1, more preferably 10 to 0.5, and even more preferably from 5 to 1 from the viewpoint of improving the rinse feel in treatment with the cleansing composition, feel in rinsing hair, and freshness in skin.

Preferably, the cleansing composition according to the present invention further contains a moisturizing agent (hereinafter, also referred to as a component (C)) from the viewpoint of improving the rinse feel in treatment with the cleansing composition, combability, softness, and manageability of hair after drying, and moist feeling in skin, and suppressing damages of hair and skin in cleansing.

Examples of preferable moisturizing agents include one or more selected from ceramides, plant extracts, sodium hyaluronate, propylene glycol, 1,3-butylene glycol, glycerol and derivatives thereof, sorbitol and derivatives thereof, sodium lactate, sodium pyrrolidonecarboxylate, carbitol, diethylene glycol monomethyl ether, sugar alcohols (such as maltitol, xylitol, sorbitol, erythritol, and lactitol), medium-chain fatty acid triglycerides, and urea, or include other moisturizing agent. Ceramides, plant extracts, sodium hyaluronate, glycerol and derivatives thereof are preferable. Examples of the derivatives of glycerol include polyoxyethylene (26) glyceryl ether.

The ceramides include natural ceramides, synthetic ceramides, and analogs thereof obtained by synthesis and the like (pseudo-ceramides). Examples of such ceramides include Ceramide H03 (Sederma), Ceramide II (Sederma), Questamide H (Quest), Ceramide TIC-001 (TAKASAGO INTERNATIONAL CORPORATION), and SOFCARE Ceramide SL-E (Kao Corporation) and the like. Particularly preferable examples of the ceramide analogs obtained by synthesis include amide derivatives including the SOFCARE Ceramide SL-E, which are represented by the general formula (3) below:

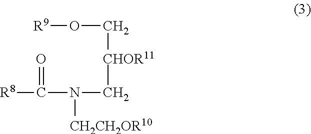

[wherein R⁸ and R⁹ may be the same or different, and represent a linear or branched saturated or unsaturated hydrocarbon group having 7 to 39 carbon atoms and optionally substituted one or more hydroxy groups; R¹⁰ and R¹¹ may be the same or different, represent a hydrogen atom, phosphoric acid salt residue, sulfuric acid salt residue, or sugar residue. It should be noted that it has one or more hydroxy groups in one molecule.]

In the general formula (3), the hydrocarbon group in R⁸ is preferably a linear or branched saturated or unsaturated hydrocarbon group having from 9 to 25 carbon atoms. The hydrocarbon group in R⁹ is preferably a linear or branched saturated or unsaturated hydrocarbon group having from 10 to 26 carbon atoms. R¹⁰ and R¹¹ are preferably a hydrogen atom.

The method for producing the amide derivative (3) is described in JP-A-62-228048, JP-A-63-216852, and the like in detail.

The ceramides may be used alone or in combination of two or more kinds. The amount of the ceramides to be blended in the cleansing composition according to the present invention is preferably from 0.001 to 30% by mass, more preferably from 0.005 to 10% by mass, and even more preferably from 0.01 to 5% by mass. Additionally, the ceramides may be any form of soluble ceramides, emulsified ceramides, liquid crystallized ceramides, and a dispersion liquid. In the case of the dispersion liquid, the ceramides are preferably those which are solid at room temperature (25° C.). From the viewpoint of stability when the cleansing composition is produced, the ceramides are preferably those which have a melting point of 30° C. or more, and more preferably a melting point of 40° C. or more. In the case of the dispersion liquid, the ceramides have an average particle size of preferably from 0.5 to 150 μm, more preferably from 1 to 150 μm, and even more preferably from 1 to 80 μm. The average particle size used therein is an arithmetic average value of values obtained as follows: a photograph is taken under transmitted light using an optical microscope; and 30 particles on the photograph are arbitrarily selected; in each of the particles, the longest portion as a linear dimension is measured.

Examples of the plant extracts include plant extracts available from ICHIMARU PHARCOS Co., Ltd. such as aloe, aloe vera, ginkgo (Ginkgo biloba), fennel (Foeniculum vulgare), seaweed, puerariae radix (Pueraria lobata), chamomile, kiwifruit (Actinidia deliciosa), cucumber, loofah (luffa), cape jasmine (Gardenia jasminoides), rice bran, peach, yuzu (Citrus junos), and hatomugi (Coix lacryma-jobi var. ma-yuen). Preferably, the amount of the plant extracts to be contained in the cleansing composition according to the present invention is preferably from 0.001 to 10% by mass, more preferably from 0.005 to 5% by mass, and even more preferably from 0.01 to 3% by mass from the viewpoint of stability and a moisturizing effect. If the cleansing composition contains the plant extracts, not only the moisturizing effect but also an anti-inflammatory effect and the like unique to the extract are given. Preferably, the amount of sodium hyaluronate as the moisturizing agent to be contained in the composition is from 0.001 to 10% by mass, more preferably from 0.005 to 5% by mass, and even more preferably from 0.01 to 3% by mass from the viewpoint of stability and the moisturizing effect.

The mass content ratio of the component (A) to the component (C) [component (A)/component (C)] is preferably from 200 to 0.01, more preferably from 50 to 0.05, and even more preferably from 20 to 0.1 from the viewpoint of developing good softness and sliding property. The content of the component (C) is preferably from 0.001 to 30% by mass, and more preferably from 0.005 to 10% by mass in the cleansing composition according to the present invention from the viewpoint of stability and the moisturizing effect. The moisturizing agents as the component (C) may be used alone or in combination of two or more kinds.

Preferably, the cleansing composition according to the present invention further contains an antibacterial agent or a fungicide (hereinafter, also referred to as a component (D)) from the viewpoint of improving the rinse feel in treatment with the cleansing composition, imparting combability and softness of hair after drying, and moist feeling of skin, improving a deodorant effect, and reducing itching after cleansing.

Examples of the antibacterial agent include triclosan, triclocarban, piroctone olamine, zinc pyrithione, selenium disulfide, 3-methyl-4-(1-methylethyl)phenol and the like, and antibacterial agents described in Koushouhin Iyakuhin Bofusakkinzainadono Kagaku (edited by John J. Cabala, Fragrance Journal Ltd.). Among these, triclosan, triclocarban, piroctone olamine, and zinc pyrithione are more preferable. In the case where the cleansing composition according to the present invention is applied to a cleansing composition for hair which is used without washing off the cleansing composition, particularly, use of triclosan, triclocarban, or piroctone olamine is preferable because the feel of hair is not impaired.

Among these components (D), examples of cationic antibacterial agents include quaternary ammonium salts represented by the general formula (4):

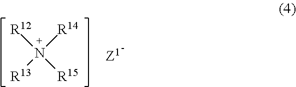

(R¹² and R¹³ represent a long-chain alkyl group, long-chain alkenyl group, or long-chain hydroxyalkyl group each having from 6 to 14 carbon atoms and from 16 to 26 carbon atoms in total, and may be the same or different from each other; R¹⁴ and R¹⁵ represent an alkyl group or hydroxyalkyl group having from 1 to 3 carbon atoms, or a polyoxyethylene group having the average number of moles added of 10 or less, and may be the same or different from each other; Z¹ represents a halogen atom, amino acid, fatty acid, an anionic residue of phosphoric acid ester, phosphonic acid ester, sulfonic acid ester, or sulfuric acid ester having a linear or branched alkyl group or alkenyl group having from 1 to 30 carbon atoms, or an anionic oligomer or polymer containing a formalin condensate of a sulfonated polycyclic aromatic compound optionally having styrene sulfonic acid having a polymerization degree of 3 or more or a hydrocarbon group as a substituent);

benzalkonium salts and benzethonium salts represented by the general formula (5):

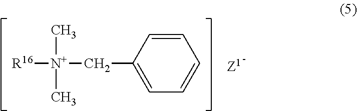

(wherein R¹⁶ represents a hydrocarbon group having from 8 to 14 carbon atoms or a group represented by:

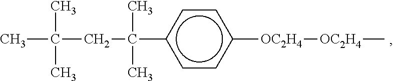

and Z¹ represents the same as above);
chlorhexidine salts represented by the general formula (6):

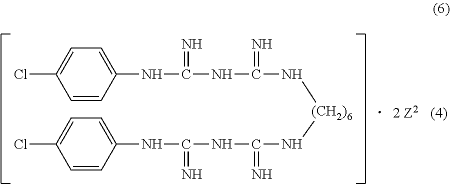

(wherein Z² represents gluconic acid, acetic acid, or hydrochloric acid);
and pyridinium salts represented by the general formula (7):

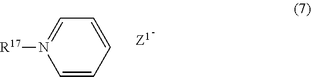

(wherein R¹⁷ represents a linear or branched alkyl group having from 6 to 18 carbon atoms, and Z¹ represents the same as above), and the like.

As Z¹ in the general formulas (4), (5), and (7) above, a halogen atom is more preferable.

Suitable specific examples of a cationic antibacterial agent include benzalkonium chloride, benzethonium chloride, cetylpyridinium chloride, chlorhexidine gluconate, chlorhexidine acetate, and chlorhexidine hydrochloride and the like. Further, those represented by the general formula (5) such as benzalkonium chloride and benzethonium chloride are preferable.

The antibacterial agents and cationic antibacterial agents as the component (D) may be used alone or in combination. Use of a combination of the antibacterial agent with the cationic antibacterial agent can provide a higher effect. The amount of the component (D) to be blended with the cleansing composition according to the present invention is preferably from 0.005 to 5% by mass, more preferably from 0.1 to 4% by mass, more preferably from 0.4 to 3% by mass, more preferably from 0.01 to 2% by mass, and even more preferably from 0.04 to 1% by mass. The mass content ratio of the component (A) to the component (D) is preferably from 100 to 0.02, more preferably from 20 to 0.05, and even more preferably from 10 to 0.1.

From the viewpoint of improving the appearance, preferably, the cleansing composition according to the present invention further contains a pearling agent (hereinafter, also referred to as a component (E)).

The pearling agent is a component that can give pearl-like gloss to the appearance of the cleansing composition. In the present invention, emulsifying agents such as esters (such as monoester and diester) of glycol and fatty acid and long-chain dialkyl ethers, water-insoluble inorganic salts, or compounds known as the pearling agent may be used. Specifically, examples of the pealing agents include organic compounds such as ethylene glycol distearate, ethylene glycol monostearate, diethylene glycol distearate, ethylene glycol dipalmitate, and distearyl ether. From the viewpoint of cost and availability, esters of glycol and fatty acid are suitable. Among these, ethylene glycol monostearate, ethylene glycol distearate are preferable, and ethylene glycol distearate is more preferable. Examples of the water-insoluble inorganic salts include titanium oxide, tin oxide, and mica and the like.

From the viewpoint of economy, cleansing property, and the appearance, the content of the component (E) is preferably not less than 0.3% by mass and not more than 10% by mass, more preferably not less than 1.0% by mass and not more than 5.0% by mass, and even more preferably from 1.0 to 4.0% by mass in the cleansing composition according to the present invention.

In the present invention, the pearling agent may be directly added to the cleansing composition for skin or hair. Alternatively, a pearl component may be crystallize in advance to prepare a premix (pearling agent mixture), and then the premix may be added to the cleansing composition.

The component (A) as a pearl crystal precipitating aid may be used for the pearling agent mixture. From the viewpoint of crystal stability of the pearling agent mixture and improvement in dispersibility when the pearling mixture is added to the cleansing composition, the mass content ratio of the component (A) to the component (E) in the pearling agent mixture [component (A)/component (E)] is preferably from 10 to 0.1, more preferably from 5 to 0.5, and even more preferably from 3 to 0.8.

From the viewpoint of improving the rinse feel in treatment with the cleansing composition, imparting combability after rising of hair and manageability after drying of hair, and moist feeling of skin, and improving a long-lasting effect of the feel, preferably, the cleansing composition according to the present invention further contains a fragrance (hereinafter, also referred to as a component (F)).

Examples of the fragrance used in the present invention include one or two or more selected from the group consisting of citrus fragrances (F1), floral fragrances (F2), woody fragrances (F3), fruity fragrances (F4), spicy fragrances (F5), musk fragrances (F6), green fragrances (F7), and others (F8). From the viewpoint of the long-lasting effect of the fragrance after use, among these, preferable fragrances are: (F1) limonene, (F2) linalool, linalyl acetate, citronellol, phenylethyl alcohol, methyl dihydrojasmonate, lyral, γ-methyl ionone, and β-ionone, (F3) ambroxan and p-t-butylcyclohexyl acetate, (F4) o-t-butylcyclohexyl acetate, damascone, and γ-undecalactone, (F5) eugenol, (F6) PEARLIDE and tentarome, and (F7) helional, tripural, menthol, and camphor. The proportion of the fragrance is preferably from 0.05 to 1.0% by mass, and more preferably from 0.1 to 0.7% by mass. The mass content ratio of the component (A) to the component (F) [component (A)/component (F)] is preferably from 100 to 0.01, more preferably from 20 to 0.1, and even more preferably from 10 to 1.

From the viewpoint of improving stability of the appearance, viscosity, smell, and the like, preferably, the cleansing composition according to the present invention further contains an organic solvent (hereinafter, also referred to as a component (G)). Examples of the organic solvent used include aromatic alcohols, carbonates, or a hydroxy compound represented by the general formula (8) below:

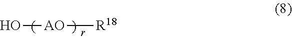

(wherein R¹⁸ represents a hydrogen atom or an linear or branched alkyl group or alkenyl group having from 1 to 4 carbon atoms; A represents an alkylene group having from 2 to 4 carbon atoms; the average number r of moles added represents the number of from 1 to 3000, the r number of A may be the same or different, and A has any disposition).

Examples of the aromatic alcohol include benzyl alcohol, benzyloxyethanol, and phenoxyethanol. Examples of carbonates include alkylene carbonates such as ethylene carbonate and propylene carbonate.

A preferable hydroxy compound represented by the general formula (8) above is those in which A is an linear or branched alkylene group having 2 or 3 carbon atoms, and more preferably a linear or branched alkylene group having 3 carbon atoms; R¹⁸ is hydrogen, a linear alkyl group or alkenyl group having from 2 to 4 carbon atoms, and more preferably hydrogen or a linear alkyl group having 2 or 3 carbon atoms; r is from 1 to 1000 (the average number of moles added), and more preferably from 1 to 100 (the average number of moles added).

Specific examples of the hydroxy compound represented by the general formula (8) include ethylene glycol, diethylene glycol, polyethylene glycol, propylene glycol, dipropylene glycol, polypropylene glycol, diethylene glycol monomethyl ether, diethylene glycol monoethyl ether, diethylene glycol monopropyl ether, and diethylene glycol monobutyl ether and the like.

A preferable organic solvent used for the present invention is benzyl alcohol, benzyloxyethanol, propylene carbonate, diethylene glycol monoethyl ether, diethylene glycol monobutyl ether, diethylene glycol, polyethylene glycol, dipropylene glycol, or polypropylene glycol, and more preferably, benzyl alcohol, diethylene glycol monoethyl ether, diethylene glycol monobutyl ether, polyethylene glycol, or polypropylene glycol from the viewpoint of improving the stability of the cleansing composition. In the case where the organic solvent is polyethylene glycol or polypropylene glycol, the weight average molecular weight (measurement method: GPC weight method) is preferably from 200 to 10000, more preferably from 200 to 4000, and even more preferably from 300 to 1500.

From the viewpoint of improvement in stability and cleansing property, the content of the component (G) is preferably from 0.1 to 30% by mass, more preferably from 0.2 to 20% by mass, and even more preferably from 0.5 to 10% by mass in the cleansing composition according to the present invention. From the viewpoint of improvement in stability and cleansing property, the mass content ratio of the component (A) to the component (G) [component (A)/component (G)] is preferably from 100 to 0.01, more preferably from 10 to 0.1, and even more preferably from 5 to 0.5.

From the viewpoint of improvement in long-lasting effect of the feel of hair and skin after drying and cares of damaged hair and skin, preferably, the cleansing composition according to the present invention further contains vitamins (hereinafter, also referred to as a component (H)).

Examples of the vitamins include vitamins A such as retinol, retinol acetate, retinol palmitate, and β-carotene; vitamins B such as pyridoxine hydrochloride (vitamin B6), nicotinic acid derivatives (nicotinic acid amide, nicotinic acid benzyl ester), γ-oryzanol, riboflavin (vitamin B2), and vitamin B derivatives such as riboflavin acetic acid ester, pyridoxine dicaprylate, and pyridoxine dipalmitate; vitamins C such as ascorbic acid (vitamin C), monostearyl ascorbate, and ascorbic acid phosphoric acid ester; vitamins D such as ergocalciferol (vitamin D2); vitamins E such as dl-tocopherol (vitamin E), tocopherol acetate, and tocopherol nicotinate; pantothenic acids such as calcium pantothenate, pantothenic acid ethyl ether, and D-pantothenyl alcohol (D-panthenol); and vitamins H such as biotin.

From the viewpoint of cares of damaged hair and skin, preferable vitamins used for the present invention are retinol, retinol palmitate, β-carotene, pyridoxine hydrochloride (vitamin B6), nicotinic acid amide, γ-oryzanol, riboflavin (vitamin B2), ascorbic acid (vitamin C), dl-tocopherol (vitamin E), tocopherol acetate, pantothenic acid ethyl ether, D-pantothenyl alcohol (D-panthenol), or biotin.

From the viewpoint of stable blending and improvement in long-lasting effect of the feel, the content of the component (H) is preferably from 0.01 to 10% by mass, more preferably from 0.05 to 5% by mass, and even more preferably from 0.1 to 3% by mass in the cleansing composition according to the present invention.

From the viewpoint of improvement in long-lasting effect of the feel of hair and skin after drying and cares of damaged hair and skin by the component (A) and the component (H), the mass content ratio of the component (A) to the component (H) [component (A)/component (H)] is preferably from 200 to 0.1, more preferably from 100 to 1, and even more preferably from 50 to 5.

From the viewpoint of improvement in stability and adjustment of viscosity, preferably, the cleansing composition according to the present invention further contains a thickener (hereinafter, also referred to as a component (I)).

Examples of the thickener include guar gum, locust bean gum, quince seed gum, carrageenan, galactan, gum arabic, tragacanth gum, pectin, mannan, starch, xanthan gum, dextran, succionoglucan, curdlan, hyaluronic acid, gelatin, casein, albumin, collagen, shellac, methyl cellulose, ethyl cellulose, hydroxyethyl cellulose, hydroxypropyl cellulose, carboxymethyl cellulose, methyl hydroxypropyl cellulose, soluble starch, carboxymethyl starch, methyl starch, hydroxypropyl starch, alginic acid propylene glycol ester, alginic acid salt, polyvinyl alcohol, polyvinylpyrrolidone, polyvinyl methyl ether, carboxyvinyl polymers, sodium polyacrylate, polyvinyl methacrylate, polyethylene oxide, ethylene oxide-propylene oxide block copolymers, polyglycol-polyamine condensates, polymethyl methacrylate fine particles, bentonite, laponite, fine powder of silicon oxide, colloidal alumina, or VEEGUM.

From the viewpoint of improvement in the stability of the cleansing composition and adjustment of viscosity, the content of the component (I) is preferably from 0.01 to 10% by mass, more preferably from 0.05 to 5% by mass, and even more preferably from 0.1 to 3% by mass in the cleansing composition according to the present invention. The mass content ratio of the component (A) to the component (I) [component (A)/component (I)] is preferably from 200 to 0.5, more preferably from 100 to 1, and even more preferably from 50 to 2.

From the viewpoint of improvement in the feel and viscosity stability, preferably, the cleansing composition according to the present invention further contains water-soluble salts (hereinafter, also referred to as a component (J)). Preferably, the water-soluble salts are one or more selected from water-soluble organic salts other than water-soluble inorganic salts and surfactants. Examples of preferable water-soluble salts include salts of organic acids such as citric acid, malic acid, succinic acid, and lactic acid; and salts of inorganic acids such as hydrochloric acid, sulfuric acid, nitric acid, carbonic acid, and phosphoric acid. Examples of cations which form the salts include alkali metals such as sodium and potassium, or ammonium and aluminum and the like. Preferable specific examples of these salts include alkali metal salts of inorganic acids such as sodium chloride and sodium sulfate; ammonium salts of inorganic acids such as ammonium chloride, ammonium sulfate, and ammonium nitrate; alkali metal salts of organic acids (particularly, aliphatic organic acids are preferable) such as trisodium citrate; and ammonium salts of organic acids (particularly, aliphatic organic acids are preferable). Among these, alkali metal salts of inorganic acids or ammonium salts of inorganic acids are preferable from the viewpoint of a use feel when these are used for a cleansing composition for skin or hair. One or more thereof may be used.

From the viewpoint of improvement in the feel and the effect of adjusting viscosity, the content of the component (J) is preferably from 0.1 to 10% by mass, more preferably from 0.2 to 8% by mass, more preferably from 0.3 to 5% by mass, and even more preferably from 0.5 to 4% by mass in the cleansing composition according to the present invention. From the viewpoint of improvement in the feel and the effect of adjusting viscosity, the mass content ratio of the component (A) to the component (J) (component (A)/component (J)) is preferably from 100 to 0.1, more preferably from 50 to 0.5, and even more preferably from 20 to 2. At a mass ratio within this range, an effect of improving fluidity can be sufficiently demonstrated.

From the viewpoint of improving the appearance and stability of the cleansing composition, and the sterilization effect and antibacterial effect, preferably, the cleansing composition according to the present invention further contains a chelating agent (hereinafter, also referred to as a component (K)).

The chelating agent used in the present invention is not particularly limited as long as the chelating agent has an ability to chelate metal ions. Examples thereof include aminopolycarboxylic acid chelating agents, aromatic and aliphatic carboxylic acid chelating agents, amino acid chelating agents, ether polycarboxylic acid chelating agents, phosphonic acid chelating agents such as iminodimethylphosphonic acid (IDP), alkyl diphosphonic acid (ADPA), and 1-hydroxyethane-1,1-diphosphonic acid (DEQUEST™ 2010), hydroxycarboxylic acid chelating agents, phosphoric acid chelating agents, polymer electrolyte (including oligomer electrolytes) chelating agents, and dimethyl glyoxime (DG). These chelating agents may be a free acid type or a salt type such as sodium salts, potassium salts, and ammonium salts. Further, these may be an ester derivative type that can be hydrolyzed.

Specific examples of the aminopolycarboxylic acid chelating agents include ethylenediaminetetraacetic acid (EDTA), cyclohexanediaminetetraacetic acid (CDTA), nitrilotriacetic acid (NTA), iminodiacetic acid (IDA), N-(2-hydroxyethyl)iminodiacetic acid (HIMDA), diethylenetriaminepentaacetic acid (DTPA), N-(2-hydroxyethyl)ethylenediaminetriacetic acid (EDTA-OH) and glycoletherdiaminetetraacetic acid (GEDTA), and salts thereof.

Specific examples of the aromatic or aliphatic carboxylic acid chelating agents include oxalic acid, malonic acid, succinic acid, glutaric acid, adipic acid, itaconic acid, aconitic acid, pyruvic acid, salicylic acid, acetylsalicylic acid, hydroxybenzoic acid, aminobenzoic acid (including anthranilic acid), phthalic acid, trimellitic acid, and gallic acid, as well as salts, methyl esters, and ethyl esters thereof. Examples of the amino acid chelating agents include glycine, serine, alanine, lysine, cystine, cysteine, ethionine, tyrosine, methionine, and salts and derivatives thereof.

Further, examples of the etherpolycarboxylic acid chelating agents include diglycolic acid, compounds represented by the general formula (9) below, and analogous compounds and salts thereof (such as sodium salts):

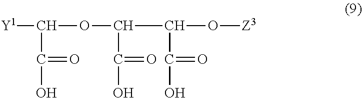

[wherein Y¹ represents a hydrogen atom, —CH₂COOH, or —COOH, and Z³ represents a hydrogen atom, —CH₂COOH, or

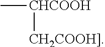

Specific examples of the hydroxycarboxylic acid chelating agents include malic acid, citric acid, glycolic acid, gluconic acid, peptonic acid, tartaric acid and lactic acid, and salts thereof. Examples of the phosphoric acid chelating agents include orthophosphoric acid, pyrophosphoric acid, triphosphoric acid, and polyphosphoric acid. Specific examples of the polymer electrolyte (including oligomer electrolytes) chelating agents include acrylic acid polymers, maleic anhydride polymers, α-hydroxyacrylic acid polymers, itaconic acid polymers, copolymers composed of two or more monomers which form these polymers, and epoxy succinic acid polymers. Further, ascorbic acid, thioglycolic acid, phytic acid, glyoxylic acid and glyoxal acid, and salts thereof may also be suitably used as the chelating agent.

From the viewpoint of availability and improvement in the stability of the cleansing composition, examples of preferable chelating agents include ethylenediaminetetraacetic acid (EDTA), succinic acid, salicylic acid, oxalic acid, lactic acid, fumaric acid, tartaric acid and 1-hydroxyethane-1,1-diphosphonic acid, and salts thereof.

The content of the component (K) is preferably from 0.1 to 10% by mass, more preferably from 0.2 to 5% by mass, and even more preferably from 0.5 to 2% by mass in the cleansing composition according to the present invention.

Further, the cleansing composition according to the present invention may contain an anti-inflammatory agent such as glycyrrhizic acid, dihydrocholesterin, or allantoin in the range in which the effect of the present invention is not impaired.

From the viewpoint of giving the combability and softness to hair and the moist feeling to skin, the cleansing composition according to the present invention preferably contains an oil solution (hereinafter, also referred to as a component (L)). Specific examples of the oil solution used in the present invention include ester oils, silicone oils, ether oils, hydrocarbon oils, higher alcohols, or carboxylic acids having an optionally substituted hydroxy group and a hydrocarbon group having from 17 to 23 carbon atoms.

Specific examples of the aforementioned ester oil include castor oil, cacao oil, mink oil, avocado oil, olive oil, sunflower oil, camellia oil, apricot kernel oil, almond oil, wheat germ oil, theobroma grandiflorum seed oil, grape seed oil, babassu oil, jojoba oil, macadamia nut oil, camellia oleifera seed oil, shea butter oil, camellia reticulata seed oil, meadowfoam oil, bees wax, lanolin, hydrogenated lanolin, octyldodecyl lanolate, caprylyl eicosenoate, diisopropyl dimerate, myristyl 2-ethylhexanoate, cetyl 2-ethylhexanoate, stearyl 2-ethylhexanoate, octyl octanoate, lauryl octanoate, myristyl octanoate, isocetyl octanoate, octyl propylheptanoate, cetostearyl isononanoate, isononyl isononanoate, isotridecyl isononanoate, methyl laurate, hexyl laurate, octyl laurate, isopropyl myristate, octyl myristate, myristyl myristate, octyldodecyl myristate, isopropyl palmitate, 2-ethylhexyl palmitate, octyl palmitate, cetyl palmitate, methyl oleate, oleyl oleate, decyl oleate, isobutyl oleate, methyl stearate, 2-ethylhexyl stearate, octyl stearate, isocetyl stearate, stearyl stearate, butyl stearate, isotridecyl stearate, isopropyl isostearate, isocetyl isostearate, isostearyl isostearate, propylene glycol isostearate, 2-ethylhexyl hydroxystearate, oleyl erucate, propanediol dicaprate, diisopropyl adipate, diethoxyethyl succinate, 2-ethylhexyl succinate, sucrose polysoyate, sucrose polybehenate, sucrose tetraisostearate, glyceryl tribehenate, triisostearin, and pentaerythrityl tetrastearate.

Among them, from the viewpoint of imparting good combability, softness and manageability to hair after drying treated with the cleansing composition, sunflower oil, avocado oil, camellia oil, macadamia nut oil, shea butter oil, octyl laurate, octyl myristate, octyldodecyl myristate, isopropyl myristate, myristyl myristate, isopropyl palmitate, 2-ethylhexyl palmitate, octyl palmitate, cetyl palmitate, methyl stearate, 2-ethylhexyl stearate, octyl stearate, isocetyl stearate, stearyl stearate, butyl stearate, and isotridecyl stearate are preferable, and one or more selected from sunflower oil, avocado oil, camellia oil, macadamia nut oil, shea butter oil, octyl laurate, octyl myristate, myristyl myristate, isopropyl palmitate, octyl palmitate, cetyl palmitate, octyl stearate, isocetyl stearate, stearyl stearate, isostearyl stearate, and isostearyl isostearate are more preferable.

Also, as the aforementioned ester oil, a hydrophobic carboxylic acid ester of dipentaerythritol may also be used. The hydrophobic carboxylic acid ester of dipentaerythritol refers to a compound obtained by subjecting dipentaerythritol to dehydration condensation with one or more hydrophobic carboxylic acids. Here, the hydrophobic carboxylic acid refers to a carboxylic acid having a hydrocarbon group having from 16 to 24 carbon atoms optionally having a hydroxyl group. Specific examples of the hydrophobic carboxylic acid include palmitic acid, stearic acid, oleic acid, isostearic acid, hydroxystearic acid, and rosin acid. From the viewpoint of availability, an ester of mixed acid of hydroxystearic acid, stearic acid, and rosin acid and dipentaerythritol is preferable.

From the viewpoint of imparting good manageability to hair after drying treated with the cleansing composition according to the present invention, and imparting moist feeling to skin, as the aforementioned silicone oil, one or more selected from dimethylpolysiloxane, dimethiconol (dimethylpolysiloxane having a hydroxyl group at the end), amino-modified silicone (dimethylpolysiloxane having an amino group within the molecule), polyether-modified silicone, glyceryl-modified silicone, amino derivative silicone, silicone wax, and silicone elastomer are preferable. From the viewpoint of the finger combability and the manageability of the hair, and dispersibility during preparation of the cleansing composition for skin or hair, the viscosity of the aforementioned silicone oil is preferably from 10 to 15 million mm²/s.

From the viewpoint of imparting good combability, softness, and manageability to hair after drying treated with the cleansing composition according to the present invention, and imparting moist feeling to skin, examples of the aforementioned ether oil include polyoxypropylene hexyl ether, polyoxypropylene octyl ether, polyoxypropylene decyl ether, polyoxypropylene lauryl ether, dihexyl ether, dioctyl ether, didecyl ether, dilauryl ether, dimyristyl ether, dicetyl ether, distearyl ether, diicosyl ether, and dibehenyl ether in which the average number of moles of propyleneoxy groups added is 3, 7, 10 or 15. Among them, polyoxypropylene hexyl ether, polyoxypropylene octyl ether, polyoxypropylene decyl ether, polyoxypropylene lauryl ether, dioctyl ether, didecyl ether, and dilauryl ether in which the average number of moles of oxypropylene added is 3 are preferable, and one or more selected from polyoxypropylene octyl ether, polyoxypropylene decyl ether, and polyoxypropylene lauryl ether in which the average number of moles of oxypropylene added is 3 are more preferable.

From the viewpoint of combability, softness, and manageability to hair after drying treated with the cleansing composition according to the present invention, and from the viewpoint of moist feeling to skin, the aforementioned hydrocarbon oil is preferably saturated or unsaturated hydrocarbon having 20 or more carbon atoms.

Specific examples of the aforementioned hydrocarbon oil include squalene, squalane, liquid paraffin, liquid isoparaffin, heavy liquid isoparaffin, α-olefin oligomer, cycloparaffin, polybutene, petroleum jelly, paraffin wax, microcrystalline wax, polyethylene wax, or ceresin. From the viewpoint of manageability of hair, squalane, squalene, liquid paraffin, or paraffin wax is preferable, and one or more selected from squalane, liquid paraffin, and paraffin wax are more preferable.

From the viewpoint of maganeability of hair after drying treated with the cleansing composition of the present invention, the aforementioned higher alcohol is preferably an alcohol having a linear or branched alkyl group or alkenyl group having from 6 to 22 carbon atoms. The number of carbon atoms in the above alkyl group or alkenyl group is more preferably from 8 to 20, and even more preferably from 12 to 18. Specific examples of the aforementioned higher alcohol include hexyl alcohol, 2-ethylhexyl alcohol, octyl alcohol, decyl alcohol, isodecyl alcohol, lauryl alcohol, myristyl alcohol, palmityl alcohol, cetyl alcohol, stearyl alcohol, isostearyl alcohol, 2-octyl dodecanol, icosyl alcohol, or behenyl alcohol.

Among them, from the viewpoint of manageability of hair and moist feeling of skin, lauryl alcohol, myristyl alcohol, cetyl alcohol, stearyl alcohol, behenyl alcohol, or 2-octyl dodecanol is preferable, of which lauryl alcohol, myristyl alcohol, cetyl alcohol, stearyl alcohol, or 2-octyl dodecanol is more preferable, and one or more selected from cetyl alcohol, stearyl alcohol, and 2-octyl dodecanol are more preferable. The hydrocarbon group of the aforementioned carboxylic acid having a hydrocarbon group having from 17 to 23 carbon atoms which may be substituted by a hydroxyl group is preferably a linear or branched alkyl group or alkenyl group. Specific examples of the carboxylic acid having a hydrocarbon group having from 17 to 23 carbon atoms which may be substituted by a hydroxyl group include stearic acid, oleic acid, isostearic acid, hydroxystearic acid, behenic acid, or rosin acid. Among them, from the viewpoint of finger combability and manageability of hair, stearic acid, oleic acid, isostearic acid, hydroxystearic acid, or behenic acid is preferable, of which oleic acid or isostearic acid is more preferable.

From the viewpoint of imparting combability, softness, and manageability to hair after drying treated with the cleansing composition, and imparting moist feeling to skin, the solubility of the aforementioned component (L) to be used in the present invention in 100 g of water at 20° C. is preferably from 0 to 1 g, more preferably from 0 to 0.5 g, and even more preferably from 0 to 0.1 g.

From the viewpoint of imparting good combability, softness, and manageability to hair after drying treated with the cleansing composition, and imparting moist feeling to skin, the content of the aforementioned component (L) is preferably from 0.01 to 30% by mass, more preferably from 0.03 to 20% by mass, and even more preferably from 0.05 to 15% by mass. Also, from the viewpoint of improving rinse feel, and obtaining combability after rinsing and drying, softness, and manageability of hair treated with the cleansing composition, and obtaining moist feeling to skin, the mass content ratio of the aforementioned component (A) to the aforementioned component (L), [Component (A)/Component (L)], is preferably from 0.2 to 1000, more preferably from 0.5 to 200, more preferably from 1 to 100, and even more preferably from 5 to 50.

From the viewpoint of imparting a good rinse feel, softness to hair during rinsing and after towel drying, and a moist feeling to skin after towel drying, the cleansing composition of the present invention preferably contains a cationic polymer or an amphoteric polymer (hereinbelow, may also be referred to as a component (M)).

From the viewpoint of finger combability and softness during rinsing of the hair according to the cleansing composition, softness after towel drying, volume of foam during washing of the skin, good rinse feel, and moist feeling after drying, examples of a preferable cationic polymer include cationic galactomannan, cationized hydroxyethyl cellulose, cationized hydroxypropyl cellulose, cationized starch, or a synthetic polymer synthesized by a radical polymerization.

The aforementioned cationic galactomannan is a polymer in which a cationic group is introduced into a galactomannan polysaccharide, and the cationic galactomannan is preferably a cationic polymer into which a quaternary nitrogen-containing substituent is introduced. The cationic galactomannan may be obtained by reacting a galactomannan polysaccharide with a cationizing agent.

From the viewpoint of the foaming performance by the cleansing composition, finger combability and softness during rinsing of the hair, softness after towel drying, volume of foam during washing of the skin, good rinse feel, and moist feeling after drying, examples of preferable cationic galactomannan to be used in the present invention include cationized tara gum, cationized locust bean gum, cationized Trigonella foenum-graecum gum, cationized guar gum, cationized cassia gum, cationized fenugreek gum, cationized honey locust gum, or cationized Brachychiton acerifolium. Among them, from the viewpoint of the finger combability by the cleansing composition and softness during rinsing of the hair, softness after towel drying, the volume of foam during washing the skin, a good rinse feel, and a moist feeling after drying, cationized tara gum, cationized locust bean gum, cationized guar gum, cationized cassia gum, or cationized fenugreek gum are more preferable.

Examples of a commercial product of the aforementioned cationized tara gum include CATINAL CTR-100 (the product of Toho Chemical Industry Co., Ltd.). Examples of a commercial product of the aforementioned cationized locust bean gum include CATINAL CLB-100 (the product of Toho Chemical Industry Co., Ltd.). Examples of a commercial product of the cationized Trigonella foenum-graecum gum include CATINAL CG-100 (the product of Toho Chemical Industry Co., Ltd.). Examples of a commercial product of the cationized guar gum include JAGUAR C-13S, JAGUAR C-14S, JAGUAR C-17, JAGUAR C-500, JAGUAR C-162, and JAGUAR EXCEL, all of which are sold by Rhodia, and N-Hance BF17, N-Hance 3215, N-Hance CCG450, N-Hance 3196, N-Hance BF13, N-Hance CG13, N-Hance CCG45, N-Hance 3000, AquaCat PF618, AquaCat CG518, and N-Hance HPCG1000, all of which are sold by Ashland Inc. Examples of a commercial product of the cationized cassia gum include Sensomer CT-250 polymer and Sensomer ST-400 polymer, both of which are sold by The Lubrizol Corporation.

In the present invention, cationized hydroxyethyl cellulose (hereinbelow, may also be referred to as “C-HEC”) refers to cellulose having a cationic group and an ethyleneoxy group. C-HEC is obtained by adding a cationic group and an ethyleneoxy group to cellulose. As the cationic group, a quaternary ammonium group is preferable.

Examples of a commercial product of C-HEC include UCARE JR125, UCARE JR400, UCARE JR30M, UCARE LR400, UCARE LR30M, SOFTCAT SL-5, SOFTCAT SL-30, SOFTCAT SL-60, SOFTCAT SL-100, SOFTCAT SX-400X, SOFTCAT SX-1300H, SOFTCAT SX-1300X, SOFTCAT SK-H, and SOFTCAT SK-MH, all of which are sold by The Dow Chemical Company.

In the present invention, cationized hydroxypropyl cellulose (hereinbelow, may also be referred to as “C-HPC”) refers to cellulose having a cationic group and a propyleneoxy group. The cationized hydroxypropyl cellulose is obtained by reacting a cationizing agent and propylene oxide with cellulose.

Aforementioned cationized starch refers to starch into which a quaternary nitrogen-containing substituent is introduced. The cationized starch is obtained by reacting a cationizing agent with starch. As a cationic group, a quaternary ammonium group is preferable. Examples of a commercial product of cationized starch include Sensomer CI-50, which is sold by The Lubrizol Corporation.

Examples of a preferable synthetic polymer to be used in the present invention include a methacryloxyalkyl quaternary ammonium salt/acrylamide copolymer, a diallyl quaternary ammonium salt/acrylamide copolymer, a diallyl quaternary ammonium salt/acrylic acid copolymer, a diallyl quaternary ammonium salt/acrylamide/acrylic acid copolymer, a methacrylamide alkyl quaternary ammonium salt/acrylic acid/acrylic acid ester copolymer, a methacrylamide alkyl quaternary ammonium salt/acrylic acid/acrylamide copolymer, or a diallyl quaternary ammonium salt/vinylpyrrolidone/vinylimidazole copolymer.

Among those polymer, from the viewpoint of foaming performance by the cleansing composition, softness, and softness to hair after towel drying, foaming performance during washing of the skin, good rinse feel, and moist feeling after drying, the polymer is more preferably a diallyl quaternary ammonium salt/acrylamide/acrylic acid copolymer, a methacrylamide alkyl quaternary ammonium salt/acrylic acid/acrylic acid ester copolymer, a methacrylamide alkyl quaternary ammonium salt/acrylic acid/acrylamide copolymer, or a diallyl quaternary ammonium salt/vinylpyrrolidone/vinylimidazole copolymer.

Examples of a commercial product of the aforementioned methacryloxyalkyl quaternary ammonium salt/acrylamide copolymer include Merquat TM5 (the product of The Lubrizol Corporation). Examples of a commercial product of the diallyl quaternary ammonium salt/acrylamide copolymer include Merquat™ 550, Merquat™ 740, Merquat™ 2200, and Merquat™ S (all are the products of The Lubrizol Corporation). Examples of a commercial product of the diallyl quaternary ammonium salt/acrylic acid copolymer include Merquat™ 280 and Merquat™ 295 (both are the products of The Lubrizol Corporation). Examples of a commercial product of the diallyl quaternary ammonium salt/acrylamide/acrylic acid copolymer include Merquat™ 3330 DRY (the product of The Lubrizol Corporation). Examples of a commercial product of the methacrylamide alkyl quaternary ammonium salt/acrylic acid/acrylic acid ester copolymer include Merquat™ 2001 (the product of The Lubrizol Corporation). Examples of a commercial product of the methacrylamide alkyl quaternary ammonium salt/acrylic acid/acrylamide copolymer include Merquat™ 2003 (the product of The Lubrizol Corporation). Examples of a commercial product of the diallyl quaternary ammonium salt/vinylpyrrolidone/vinyl imidazole copolymer include Luviquat® Sensation, which is manufactured and sold by BASF.

From the viewpoint of the foaming performance of the cleansing composition, softness, softness after towel dry, foaming performance during cleansing skin, good rinse feel, and moist feeling after dry, the content of the component (M) is preferably from 0.01 to 5% by mass, more preferably from 0.02 to 1% by mass, and even more preferably from 0.05 to 0.5% by mass in the cleansing composition according to the present invention. From the viewpoint of the foaming performance of the cleansing composition, softness, softness after towel dry, foaming performance during cleansing skin, good rinse feel, and moist feeling after dry, the mass content ratio of the component (A) to the component (M) [component (A)/component (M)] is preferably from 5 to 250, more preferably from 10 to 100, and even more preferably from 20 to 50 in the cleansing composition according to the present invention.

### <Other Components>

In the cleansing composition according to the present invention, a non-reacted raw material internal olefin may remain because of the production step of the component (A). The content of the raw material internal olefin in the cleansing composition according to the present invention is preferably 2.0% by mass or less, more preferably 1.0% by mass or less, more preferably 0.5% by mass or less, and even more preferably 0.2% by mass or less from the viewpoint of the volume of foam, a lathering speed, and rinse feel. The content of the raw material internal olefin in the cleansing composition according to the present invention is preferably 0.0001% by mass or more, more preferably 0.001% by mass or more, and even more preferably 0.01% by mass or more. The content of the non-reacted raw material internal olefin in the cleansing composition according to the present invention may be measured in accordance with the method described in Examples.

The raw material internal olefin used in production of the component (A) may contain a paraffin component. In this case, the paraffin component is contained in the cleansing composition according to the present invention. The content of the paraffin component is preferably 10% by mass or less, more preferably 5% by mass or less, even more preferably 3% by mass or less in the raw material internal olefin from the viewpoint of lathering property, and preferably 0.001% by mass or more in the raw material internal olefin from the viewpoint of cost reduction and improvement in productivity. The content of the paraffin component may be measured by GC-MS, for example. Specifically, the content may be determined in accordance with the method described in Examples.

The cleansing composition of the present invention may contain, in addition to the aforementioned components, water, which may serve as a medium of the component (A), a viscosity reducing agent, polyhydric alcohols, a preservative, and a reducing agent, and also, other components used as ordinary cosmetic raw materials. Examples of other components usually used as a cosmetic raw material include a feel improver, an ultraviolet absorber, a visible light absorber, an antioxidant, a colorant, a preservative, a pH adjuster, and a viscosity regulator and the like.

<Production Method of the Cleansing Composition of the Present Invention>

No particular limitation is imposed on the production method of the cleansing composition of the present invention, and it may be produced by a conventional method. Specifically, for example, in the case of a liquid shampoo for hair, water and an internal olefin sulfonate (A) are heated and mixed to homogeneity. If necessary, the internal olefin sulfonate (A) may be dispersed or dissolved in water in advance, and then added. The cleansing composition of the present invention may also be prepared by adding the internal olefin sulfonate (A) to an aqueous solution of a surfactant and homogeneously dissolving or dispersing it, followed by cooling, and if necessary, adding the aforementioned components from (B) to (M), a pH adjuster, a dye, and the like.

No particular limitation is imposed on the form of the cleansing composition of the present invention, and it may be provided in any form such as a liquid, a foam, a paste, a cream, a solid, and a powder, among which a liquid, a paste, or a cream is preferable, and a liquid is more preferable. When the cleansing composition is provided as a liquid, polyethylene glycol, ethanol, and the like are preferably used as a liquid medium in addition to water. The content of water in the cleansing composition of the present invention is preferably 10% by mass or more and 90% by mass or less.

### <Intended Use and Method of Use>

The cleansing composition of the present invention can impart a good rinse feel, combability and softness after rinsing, and manageability after drying to hair, and a moist feeling to skin; therefore, it can be preferably used as a cleansing composition for hair or a cleansing composition for skin. Examples of the cleansing composition for hair include a hair shampoo. Examples of the cleansing composition for skin include a body shampoo, a facial cleanser, a makeup remover, or a hand soap.

Because the cleansing composition of the present invention can impart a good rinse feel, combability and softness after rinsing, and manageability after drying to hair, and a moist feeling to skin, a method for washing the hair which includes applying the aforementioned cleansing composition of the present invention to hair, followed by washing and then rinsing is also provided. Also, a method for washing the body which includes applying the aforementioned cleansing composition of the present invention to a surface of the skin, followed by washing and then rinsing is also provided.

Hereinafter, with reference to the above-mentioned embodiment, the present invention will disclose the following cleansing composition for skin or hair.

[1] A cleansing composition for skin or hair comprising an internal olefin sulfonate (A) having 12 or more and 24 or less carbon atoms.
[2] The cleansing composition for skin or hair of above [1], in which the number of carbon atoms in the internal olefin sulfonate is preferably 14 or more, more preferably 16 or more, and preferably 20 or less, more preferably 18 or less.
[3] The cleansing composition for skin or hair of above [1] or [2], in which the mass content ratio of the internal olefin sulfonate having 16 carbon atoms to the internal olefin sulfonate having 18 carbon atoms in the internal olefin sulfonate (A) (internal olefin sulfonate having 16 carbon atoms/internal olefin sulfonate having 18 carbon atoms) is preferably from 50/50 to 99/1, more preferably 60/40 to 95/5, more preferably 70/30 to 90/10, more preferably 75/25 to 90/10, even more preferably 75/25 to 85/15.
[4] The cleansing composition for skin or hair of any one of above [1] to [3], in which a total content of the internal olefin sulfonate having 16 carbon atoms and the internal olefin sulfonate having 18 carbon atoms in the internal olefin sulfonate (A) is preferably 50% by mass or more, more preferably 60% by mass, more preferably 70% by mass, more preferably 80% by mass or more, more preferably 90% by mass or more, and even more preferably 95% by mass or more.
[5] The cleansing composition for skin or hair of any one of above [1] to [4], in which a content of an internal olefin sulfonate in which a sulfonate group is present at a C-2 position in the internal olefin sulfonate (A) is preferably 20% by mass or less, more preferably less than 20% by mass, more preferably 19% by mass or less, more preferably less than 18% by mass, even more preferably 17.5% by mass or less, and preferably 5% by mass or more, more preferably 6% by mass or more, more preferably 7% by mass or more, more preferably 8% by mass or more, and even more preferably 9% by mass or more.
[6] The cleansing composition for skin or hair of any one of above [1] to [5], in which the content of an olefin sulfonate in which the sulfonate group is present at the C-1 position of the olefin chain or alkane chain in the component (A) is preferably 3.0% by mass or less, more preferably 2.5% by mass or less, more preferably 2.0% by mass or less, more preferably 1.5% by mass or less, and even more preferably 1.0% by mass or less, and more preferably 0.01% by mass or more.
[7] The cleansing composition for skin or hair of any one of above [1] to [6], in which the mass content ratio (hydroxy form/olefin form) of the hydroxy form of an internal olefin sulfonate to the olefin form of an internal olefin sulfonate in the component (A) is preferably from 50/50 to 100/0, more preferably from 60/40 to 100/0, more preferably from 70/30 to 100/0, more preferably from 75/25 to 100/0, and even more preferably from 75/25 to 95/5.
[8] The cleansing composition for skin or hair of any one of above [1] to [7], in which the content of the component (A) in the cleansing composition is preferably 0.1% by mass or more, more preferably 1% by mass or more, more preferably 2% by mass or more, and even more preferably 5% by mass or more, and preferably 80% by mass or less, more preferably 50% by mass or less, more preferably 30% by mass or less, and even more preferably 20% by mass or less.
[9] The cleansing composition for skin or hair of any one of above [1] to [8], in which further comprise a surfactant other than the internal olefin sulfonate (A).
[10] The cleansing composition for skin or hair of above [9], in which the component (B) preferably includes an anionic surfactant, a nonionic surfactant, an amphoteric surfactant and a cationic surfactant other than the component (A).
[11] The cleansing composition for skin or hair of above [9] or [10], in which a content of the component (B) is preferably 50% by mass or less, more preferably 30% by mass or less, more preferably 20% by mass or less, more preferably 15% by mass or less, more preferably 10% by mass or less, more preferably 5% by mass or less, and preferably 0.5% by mass or more, more preferably 1% by mass or more, and more preferably 2% by mass or more.
[12] The cleansing composition for skin or hair of any one of above [1] to [11], in which further includes a moisturizing agent (C).
[13] The cleansing composition for skin or hair of [12], in which the component (C) is preferably one or more selected from ceramides, plant extracts, sodium hyaluronate, propylene glycol, 1,3-butylene glycol, glycerol and derivatives thereof, sorbitol and derivatives thereof, sodium lactate, sodium pyrrolidonecarboxylate, carbitol, diethylene glycol monomethyl ether, sugar alcohols (such as maltitol, xylitol, sorbitol, erythritol, and lactitol), medium-chain fatty acid triglycerides, and urea, more preferably ceramides, plant extracts, sodium hyaluronate, glycerol and derivatives thereof.
[14] The cleansing composition for skin or hair of above [12] or [13], in which the mass content ratio of the component (A) to the component (C) [component (A)/component (C)] is preferably from 200 to 0.01, more preferably from 50 to 0.05, more preferably from 20 to 0.1, more preferably from 0.001 to 30% by mass, more preferably from 0.005 to 10% by mass.
[15] The cleansing composition for skin or hair of any one of above [1] to [16], in which further includes an antibacterial agent or a fungicide (D).
[16] The cleansing composition for skin or hair of [15], in which the antibacterial agent is preferably one or two or more selected from triclosan, triclocarban, piroctone olamine, zinc pyrithione, selenium disulfide, and 3-methyl-4-(1-methylethyl)phenol.
[17] The cleansing composition for skin or hair of above [15] or [16], in which the amount of the component (D) to be blended is preferably from 0.005 to 5% by mass, more preferably from 0.1 to 4% by mass, more preferably from 0.4 to 3% by mass, more preferably from 0.01 to 2% by mass, and even more preferably from 0.04 to 1% by mass.
[18] The cleansing composition for skin or hair of any one of above [1] to [17], in which further comprises a pearling agent (E).
[19] The cleansing composition for skin or hair of above [18], in which the component (E) is preferably one or two or more selected from ethylene glycol distearate, ethylene glycol monostearate, diethylene glycol distearate, ethylene glycol dipalmitate, and distearyl ether.
[20] The cleansing composition for skin or hair of above [18] or [19], in which the content of the component (E) is preferably not less than 0.3% by mass and not more than 10% by mass, more preferably not less than 1.0 and not more than 5.0% by mass, and even more preferably from 1.0 to 4.0% by mass.
[21] The cleansing composition for skin or hair of any one of above [1] to [20], in which further includes a fragrance (F).
[22] The cleansing composition for skin or hair of above [21], in which the component (F) is preferably one or two or more selected from the group consisting of citrus fragrances (F1), floral fragrances (F2), woody fragrances (F3), fruity fragrances (F4), spicy fragrances (F5), musk fragrances (F6), green fragrances (F7) and others (F8).
[23] The cleansing composition for skin or hair of above [21] or [22], in which the mass content ratio of the component (A) to the component (F) [component (A)/component (F)] is preferably from 100 to 0.01, more preferably from 20 to 0.1, and even more preferably from 10 to 1.
[24] The cleansing composition for skin or hair of any one of above [1] to [23], in which further comprises an organic solvent (G).
[25] The cleansing composition for skin or hair of above [24], in which the component (G) preferably includes aromatic alcohols, carbonates, or a hydroxy compound represented by the general formula (8) below:

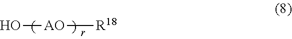

(wherein R¹⁸ represents a hydrogen atom or an linear or branched alkyl group or alkenyl group having from 1 to 4 carbon atoms; A represents an alkylene group having from 2 to 4 carbon atoms; the average number r of moles added represents the number of from 1 to 3000, the r number of A's may be the same or different, and A's have any disposition).
[26] The cleansing composition for skin or hair of above [24] or [25], in which the content of the component (G) is preferably from 0.1 to 30% by mass, more preferably from 0.2 to 20% by mass, and even more preferably from 0.5 to 10% by mass.
[27] The cleansing composition for skin or hair of any one of above [1] to [26], in which further comprises a vitamin agent (H).
[28] The cleansing composition for skin or hair of above [27], in which the component (H) preferably includes retinol, retinol palmitate, β-carotene, pyridoxine hydrochloride (vitamin B6), nicotinic acid amide, γ-oryzanol, riboflavin (vitamin B2), ascorbic acid (vitamin C), dl-tocopherol (vitamin E), tocopherol acetate, pantothenic acid ethyl ether, D-pantothenyl alcohol (D-panthenol) or biotin.
[29] The cleansing composition for skin or hair of above [27] or [28], in which the content of the component (H) is preferably from 0.01 to 10% by mass, more preferably from 0.05 to 5% by mass, and even more preferably from 0.1 to 3% by mass.
[30] The cleansing composition for skin or hair of any one of above [1] to [29], in which further comprises a thickener (I).
[30] The cleansing composition for skin or hair of above [30], in which the component (I) preferably includes guar gum, locust bean gum, quince seed gum, carrageenan, galactan, gum arabic, tragacanth gum, pectin, mannan, starch, xanthan gum, dextran, succionoglucan, curdlan, hyaluronic acid, gelatin, casein, albumin, collagen, shellac, methyl cellulose, ethyl cellulose, hydroxyethyl cellulose, hydroxypropyl cellulose, carboxymethyl cellulose, methyl hydroxypropyl cellulose, soluble starch, carboxymethyl starch, methyl starch, hydroxypropyl starch, alginic acid propylene glycol ester, alginic acid salt, polyvinyl alcohol, polyvinylpyrrolidone, polyvinyl methyl ether, carboxyvinyl polymers, sodium polyacrylate, polyvinyl methacrylate, polyethylene oxide, ethylene oxidepropylene oxide block copolymers, polyglycol-polyamine condensates, polymethyl methacrylate fine particles, bentonite, laponite, fine powder of silicon oxide, colloidal alumina or VEEGUM.
[32] The cleansing composition for skin or hair of above [30] or [31], in which the content of the component (I) is preferably from 0.01 to 10% by mass, more preferably from 0.05 to 5% by mass, and even more preferably from 0.1 to 3% by mass.
[33] The cleansing composition for skin or hair of any one of above [1] to [32], in which further comprises water-soluble salts (J).
[34] The cleansing composition for skin or hair of above [33], in which the component (J) preferably includes salts of organic acids such as citric acid, malic acid, succinic acid and lactic acid; or salts of inorganic acids such as hydrochloric acid, sulfuric acid, nitric acid, carbonic acid, and phosphoric acid.
[35] The cleansing composition for skin or hair of above [33] or [34], in which the content of the component (J) is preferably from 0.1 to 10% by mass, more preferably from 0.2 to 8% by mass, more preferably from 0.3 to 5% by mass, and even more preferably from 0.5 to 4% by mass.
[36] The cleansing composition for skin or hair of any one of above [1] to [35], in which further includes a chelating agent (K).
[37] The cleansing composition for skin or hair of above [36], in which the component (K) preferably includes aminopolycarboxylic acid chelating agents, aromatic and aliphatic carboxylic acid chelating agents, amino acid chelating agents, ether polycarboxylic acid chelating agents, phosphonic acid chelating agents such as iminodimethylphosphonic acid (IDP), alkyl diphosphonic acid (ADPA), and 1-hydroxyethane-1,1-diphosphonic acid (DEQUEST™ 2010), hydroxycarboxylic acid chelating agents, phosphoric acid chelating agents, polymer electrolyte (including oligomer electrolytes) chelating agents, and dimethyl glyoxime (DG).
[38] The cleansing composition for skin or hair of above [36] or [37], in which the content of the component (K) is preferably from 0.1 to 10% by mass, more preferably from 0.2 to 5% by mass, and even more preferably from 0.5 to 2% by mass.
[39] The cleansing composition for skin or hair of any one of above [1] to [38], in which further comprises an oil solution (L).
[40] The cleansing composition for skin or hair of above [39], in which the component (L) preferably includes ester oils, silicone oils, ether oils, hydrocarbon oils, higher alcohols, or carboxylic acids having an optionally substituted hydroxy group and a hydrocarbon group having from 17 to 23 carbon atoms.
[41] The cleansing composition for skin or hair of above [39] or [40], in which the content of the component (L) is preferably from 0.01 to 30% by mass, more preferably from 0.03 to 20% by mass, and even more preferably from 0.05 to 15% by mass.
[42] The cleansing composition for skin or hair of any one of above [1] to [41], in which further comprises a cationic polymer or an amphoteric polymer (M).
[43] The cleansing composition for skin or hair of above [42], in which the cationic polymer preferably includes cationic galactomannan, cationized hydroxyethyl cellulose, cationized hydroxypropyl cellulose, cationized starch, or a synthetic polymer synthesized by a radical polymerization.
[44] The cleansing composition for skin or hair of above [42] or [43], in which the content of the component (M) is preferably from 0.01 to 5% by mass, more preferably from 0.02 to 1% by mass, and even more preferably from 0.05 to 0.5% by mass.

### EXAMPLES

Hereinbelow, the present invention will be specifically described with reference to Examples. It should be noted that unless otherwise specifically noted, “part” means “part by mass” and “%” means “% by mass” in the following Examples and Comparative Examples. Also, the methods used for measuring various physical property are as follows.

### (1) Conditions of Measurement

(i) Method for Measuring the Position of a Double Bond in the Internal Olefin

The position of a double bond in an internal olefin was measured by gas chromatography (hereinbelow, abbreviated as GC). Specifically, an internal olefin was converted to a dithiated derivative by reaction with dimethyl disulfide, and each component was separated by GC. As a result, the position of a double bond in an internal olefin was found based on the peak area of each component.

The apparatus and analytical conditions used for the measurement are as follows. GC apparatus (trade name: HP6890, the product of Hewlett-Packard Company); Column (trade name: Ultra-Alloy-1HT capillary column, 30 m×250 μm×0.15 μm, the product of Frontier Laboratories Ltd.); Detector (flame ionization detector (FID)); Injection temperature of 300° C.; Detector temperature of 350° C.; and He flow rate of 4.6 mL/minute.

(ii) Method for Measuring the Mass Ratio of Hydroxy Form/Olefin Form

The mass ratio of hydroxy form/olefin form was measured by HPLC-MS. Specifically, the hydroxy form and the olefin form were separated by HPLC and each form was identified by separately analyzing with MS. As a result, from the resulting GC-MS peak area, the fraction of each form was obtained.

The apparatus and analytical conditions used for the measurement are as follows. HPLC apparatus (trade name: Agilent technology 1100, the product of Agilent Technologies, Inc.); Column (trade name: L-column ODS 4.6×150 mm, the product of Chemicals Evaluation and Research Institute, Japan); Sample preparation (diluted 1000-fold with methanol); Eluent A (10 mM ammonium acetate in water); Eluent B (10 mM ammonium acetate in methanol), Gradient (0 minute (A/B=30/70%)→10 minutes (30/70%)→55 minutes (0/100%)→65 minutes (0/100%)→66 minutes (30/70%)→75 minutes (30/70%); MS apparatus (trade name: Agilent technology 1100 MS SL (G1946D); and MS detection (anion detection m/z 60-1600, UV 240 nm)

(iii) Method for Measuring the Content of the Raw Material Internal Olefin

The content of the raw material internal olefin was measured by GC. Specifically, ethanol and petroleum ether were added to an aqueous solution of internal olefin sulfonate, followed by extraction to give olefin in the petroleum ether phase. As a result, from the GC peak area of the olefin, the amount thereof was quantitated. The apparatus and analytical conditions used for the measurement are as follows. GC apparatus (trade name: Agilent technology 6850, the product of Agilent Technologies, Inc.); Column (trade name: Ultra-Alloy-1HT capillary column, 15 m×250 μm×0.15 μm, the product of Frontier Laboratories, Ltd.); Detector (flame ionization detector (FID)); Injection temperature of 300° C.; Detector temperature of 350° C.; and He flow rate of 3.8 mL/minute.

(iv) Method for Measuring the Content of Inorganic Compounds

The content of inorganic compounds was measured by potentiometric titration and neutralization titration. Specifically, the content of Na₂SO₄ was quantitated by measuring sulfate ion (SO₄²⁻) by potentiometric titration. Also, the content of NaOH was quantitated by neutralization titration with diluted hydrochloric acid.

(vi) Method for Measuring the Content of the Paraffin Component

The content of the paraffin component was measured by GC. Specifically, ethanol and petroleum ether were added to an aqueous solution of internal olefin sulfonate, followed by extraction to give paraffin in the petroleum ether phase. As a result, from the GC peak area of the paraffin, the amount thereof was quantitated. It should be noted that the apparatus and analytical conditions used for measurement are the same as those used for the measurement of the content of the raw material internal olefin.

### (2) Production of an Internal Olefin

### Production Example A

Into a flask with a stirrer, 7000 g (25.9 moles) of 1-octadecanol (trade name: KALCOL 8098, the product of Kao Corporation), and as a solid acid catalyst, 1050 g (15 wt % relative to the raw material alcohol) of γ-alumina (STREM Chemicals, Inc.) were placed, and reactions were allowed to proceed for 13 hours at 285° C. while stirring and passing nitrogen (7000 mL/min.) through the system. The alcohol conversion ratio was 100% and the purity of C18 internal olefin was 98.5% after the completion of the reaction. The resulting crude internal olefin was transferred to a distillation flask and distilled at from 148 to 158° C./0.5 mmHg, whereby 100% pure internal olefin having 18 carbon atoms was obtained. The double bond distribution in the resulting internal olefin was as follows; C-1 position, 0.7% by mass; C-2 position, 16.9% by mass; C-3 position, 15.9% by mass; C-4 position, 16.0% by mass; C-5 position, 14.7% by mass; C-6 position 11.2% by mass; C-7 position, 10.2% by mass; and C-8 and 9 positions, 14.6% by mass in total.

### Production Example B

Into a flask with a stirrer, 7000 g (28.9 moles) of 1-hexadecanol (trade name: KALCOL 6098, the product of Kao Corporation), and as a solid acid catalyst, 700 g (10% by mass relative to the raw material alcohol) of γ-alumina (STREM Chemicals, Inc.) were placed, and reactions were allowed to proceed for five hours at 280° C. while stirring and passing nitrogen (7000 mL/minute) through the system. The alcohol conversion ratio was 100%, and the purity of C16 internal olefin was 99.7% after the completion of the reaction. The resulting crude internal olefin was transferred to a distillation flask and distilled at from 136 to 160° C./4.0 mmHg, whereby 100% pure internal olefin having 16 carbon atoms was obtained. The double bond distribution in the resulting internal olefin was as follows; C-1 position, 0.5% by mass; C-2 position, 16.5% by mass; C-3 position, 15.4% by mass; C-4 position, 16.4% by mass; C-5 position, 17.2% by mass; C-6 position, 14.2% by mass; and C-7 and 8 positions, 19.8% by mass in total.

### Production Example C

Into a flask with a stirrer, 7000 g (28.9 moles) of 1-hexadecanol (trade name: KALCOL 6098, the product of Kao Corporation), and as a solid acid catalyst, 700 g (10 wt % relative to the raw material alcohol) of γ-alumina (STREM Chemicals, Inc.) were placed, and reactions were allowed to proceed for three hours at 280° C. while stirring and passing nitrogen (7000 mL/minute) through the system. The alcohol conversion ratio was 100% and the purity of C16 internal olefin was 99.6% after the completion of the reaction. The resulting crude internal olefin was transferred to a distillation flask and distilled at from 136 to 160° C./4.0 mmHg, whereby 100% pure internal olefin having 16 carbon atoms was obtained. The double bond distribution in the resulting internal olefin was as follows; C-1 position, 1.8% by mass; C-2 position, 30.4% by mass; C-3 position, 23.9% by mass; C-4 position, 16.8% by mass; C-5 position, 12.0% by mass; C-6 position, 7.4% by mass; and C-7 and 8 positions, 7.8% by mass in total.

### Production Example D

Into a flask with a stirrer, 7000 g (25.9 moles) of 1-octadecanol (trade name: KALCOL 8098, the product of Kao Corporation), and as a solid acid catalyst, 700 g (10 wt % relative to the raw material alcohol) of γ-alumina (STREM Chemicals, Inc.) were placed, and reactions were allowed to proceed for 10 hours at 280° C. while stirring and passing nitrogen (7000 mL/minute) through the system. The alcohol conversion ratio was 100% and the purity of C18 internal olefin was 98.2% after the completion of the reaction. The resulting crude internal olefin was transferred to a distillation flask and distilled at the temperature inside of from 148 to 158° C./0.5 mmHg, whereby 100% pure purified internal olefin was obtained. The double bond distribution in the resulting internal olefin was as follows; C-1 position, 0.8% by mass; C-2 position, 31.3% by mass; C-3 position, 22.9% by mass; C-4 position, 15.5% by mass; C-5 position, 10.8% by mass; C-6 position, 7.2% by mass; C-7 position, 5.3% by mass; and C-8 and 9 positions, 6.2% by mass in total.

### (3) Production of an Internal Olefin Sulfonate

### Production Example 1

Using a thin film sulfonation reactor (14 mm in inner diameter and 4 m in length), the sulfonation reaction of the internal olefin having 16 carbon atoms produced in Production Example C was carried out by passing through sulfur trioxide gas containing a concentration of SO₃ at 2.8% by volume, while passing cooling water of 20° C. through the outer jacket of the reactor. It should be noted that the reaction molar ratio of SO₃/internal olefin was set at 1.09.

The resulting sulfonation product was added to an alkaline aqueous solution containing 1.2 times the molar amount of sodium hydroxide relative to the theoretical acid value (AV), followed by neutralization at 30° C. for one hour while stirring. The resulting neutralized product was hydrolyzed by heating at 160° C. for one hour in an autoclave, whereby a crude product of sodium C16 internal olefin sulfonate was obtained.

Then, 300 g of the crude product thus obtained was transferred to a separatory funnel, to which 300 mL of ethanol was added. Then, 300 mL of petroleum ether was added per operation, whereby oil-soluble impurities were removed by extraction. At this time, inorganic compounds (mainly composed of sodium sulfate) which were precipitated at the oil-water interface by the addition of ethanol were also separated and removed from the aqueous phase by the oil-water separation operation. The above operation was repeated three times. Then, the aqueous phase side was evaporated to dryness, whereby sodium internal olefin sulfonate having 16 carbon atoms was obtained. The content of the raw material internal olefin was less than 100 ppm (below the GC detection limit) and that of inorganic compounds was 1.9% by mass. The above results are shown in Table 1.

### Production Example 2

Except for using the internal olefin having 18 carbon atoms produced in Production Example D, sodium internal olefin sulfonate having 18 carbon atoms was obtained under the same conditions as those used in Example 1. The content of the raw material internal olefin was less than 100 ppm (below the GC detection limit) and that of inorganic compounds was 0.9% by mass. The above results are shown in Table 1.

### Production Example 3

Except for using the internal olefin having 16 carbon atoms produced in Production Example B, sodium internal olefin sulfonate having 16 carbon atoms was obtained under the same conditions as those used in Example 1. The content of the raw material internal olefin was less than 100 ppm (below the GC detection limit) and that of inorganic compounds was 1.3% by mass. The above results are shown in Table 1.

### Production Example 4

Except for using the internal olefin having 18 carbon atoms produced in Production Example A, sodium internal olefin sulfonate having 18 carbon atoms was obtained under the same conditions as those used in Example 1. The content of the raw material internal olefin was less than 100 ppm (below the GC detection limit) and that of inorganic compounds was 1.7% by mass. The above results are shown in Table 1.

TABLE 1

| | | | Internal olefin |
|---|---|---|---|
| | Raw material internal olefin | | sulfonate |
| | Number of | C-2 Double | HAS/IOS |
| | carbon atoms | bond (%) | (Mass ratio) |
| Production example 1 | C16 | 30.4 | 80/20 |
| Production example 2 | C18 | 31.3 | 80/20 |
| Production example 3 | C16 | 16.5 | 80/20 |
| Production example 4 | C18 | 16.9 | 80/20 |

### (4) Preparation of the Cleansing Compositions

Using internal olefin sulfonates shown in Table 1, the cleansing compositions for hair or skin each having the compositions shown in from Tables 2 to 5 were prepared by a conventional method. Specifically, the component (A) and appropriate amounts of water, and the components (B) to (M) if necessary, were taken in a beaker. The resulting mixture were heated to 60° C. and mixed, and then cooled to room temperature. Then, the mixture was supplemented with water and adjusted to pH 6 with a pH adjuster (a 50% aqueous solution of citric acid or a 10% aqueous solution of sodium hydroxide), whereby each cleansing composition was obtained.

TABLE 2

| | | Formula Examples | | | | | | | | | |
|---|---|---|---|---|---|---|---|---|---|---|---|
| Cleansing composition | | 1 | 2 | 3 | 4 | 5 | 6 | 7 | 8 | 9 | 10 |
| (A) | Internal olefin sulfonate (1) | 4.8 | 8.0 | 8.0 | 8.0 | 8.0 | 8.0 | 8.0 | 8.0 | 8.0 | 8.0 |
| | Internal olefin sulfonate (2) | 1.2 | 2.0 | 2.0 | 2.0 | 2.0 | 2.0 | 2.0 | 2.0 | 2.0 | 2.0 |
| (B) | Sodium laurylethersulfate*¹ | 6.0 | | | | | | | | | |
| | Sodium lauryl sulfate*² | | | | | | | | | | |
| (C) | Pseudo ceramide*³ | | 0.3 | | | | | | | | |
| | Plant extracts*⁴ | | | | | | | | | | |
| (D) | Triclosan*⁵ | | | 0.2 | | | | | | | |
| | Benzalkonium chloride*⁶ | | | | | | | | | | |
| (E) | Ethylene glycol distearate*⁷ | | | | 1.0 | | | | | | |
| | Mixture of ethylene glycol distearate*⁸ | | | | | | | | | | |
| (F) | Limonene*⁹ | | | | | 0.3 | | | | | |
| | Menthol*¹⁰ | | | | | | | | | | |
| (G) | Polypropylene glycol*¹¹ | | | | | | 1.0 | | | | |
| | Benzyl alcohol*¹² | | | | | | | | | | |
| (H) | Riboflavin*¹³ | | | | | | | | | | |
| | D-panthenol*¹⁴ | | | | | | | 0.1 | | | |
| (I) | Carboxyvinyl polymer*¹⁵ | | | | | | | | | | |
| | Hydroxyethyl cellulose*¹⁶ | | | | | | | | 1.0 | | |
| (J) | Sodium chloride | | | | | | | | | | |
| | Sodium sulfate | | | | | | | | | 0.3 | |
| (K) | EDTA | | | | | | | | | | |
| | HEDP | | | | | | | | | | 0.5 |

| | |
|---|---|
| pH adjuster | q.s. |
| Water | Balance |

*¹made by Kao Corporation, trade name: EMAL 270J (effective component of 70%)
*²made by Kao Corporation, trade name: EMAL 30NS
*³made by Kao Corporation, trade name: SOFCARE Ceramide SL-E
*⁴made by ICHIMARU PHARCOS Co., Ltd., trade name: aloe vera extract (effective component of 1%)
*⁵made by Ciba-Geigy AG, trade name: Irgasan DP-300
*⁶made by Kao Corporation, trade name: SANISOL C (effective component of 50%)
*⁷made by Kao Corporation, trade name: EMANON 3201M-V
*⁸made by Cognis GmbH, trade name: Euperlan PK-3000 (effective component of 20%)
*⁹made by Yamakei Sangyo K.K.
*¹⁰made by The Suzuki Menthol Co., Ltd., trade name: 1-mentho1
*¹¹made by Sanyo Chemical Industries, Ltd., trade name: NEWPOL PP-1000
*¹²made by TAKASAGO INTERNATIONAL CORPORATION
*¹³made by DSM Nutritional Japan K.K.
*¹⁴made by BASF SE, trade name: D-panthenol 50P (effective component of 50%)
*¹⁵made by Lubrizol Advanced Materials, Inc., trade name: Carbopol 980
*¹⁶made by DAICEL CHEMICAL INDUSTRIES, LTD., trade name: HEC DAICEL HE850

TABLE 3

| | | | Formula Examples | | | | | | | | | | | | | | | | | | | |
|---|---|---|---|---|---|---|---|---|---|---|---|---|---|---|---|---|---|---|---|---|---|---|
| Cleansing composition | | | 11 | 12 | 13 | 14 | 15 | 16 | 17 | 18 | 19 | 20 | 21 | 22 | 23 | 24 | 25 | 26 | 27 | 28 | 29 | 30 |
| Blended | (A) | Internal olefin sulfonate (3) | 4.8 | 4.8 | 8.0 | 8.0 | 8.0 | 8.0 | 8.0 | 8.0 | 8.0 | 8.0 | 8.0 | 8.0 | 8.0 | 8.0 | 8.0 | 8.0 | 8.0 | 8.0 | 8.0 | 8.0 |
| composition | | Internal olefin sulfonate (4) | 1.2 | 1.2 | 2.0 | 2.0 | 2.0 | 2.0 | 2.0 | 2.0 | 2.0 | 2.0 | 2.0 | 2.0 | 2.0 | 2.0 | 2.0 | 2.0 | 2.0 | 2.0 | 2.0 | 2.0 |
| (parts by | (B) | Sodium laurylethersulfate*¹ | 6.0 | | | | | | | | | | | | | | | | | | | |
| mass) | | Sodium lauryl sulfate*² | | 6.0 | | | | | | | | | | | | | | | | | | |
| | (C) | Pseudo ceramide*³ | | | 0.3 | | | | | | | | | | | | | | | | | |
| | | Plant extracts*⁴ | | | | 0.1 | | | | | | | | | | | | | | | | |
| | (D) | Triclosan*⁵ | | | | | 0.2 | | | | | | | | | | | | | | | |
| | | Benzalkonium chloride*⁶ | | | | | | 0.5 | | | | | | | | | | | | | | |
| | (E) | Ethylene glycol distearate*⁷ | | | | | | | 1.0 | | | | | | | | | | | | | |
| | | Mixture of ethylene glycol | | | | | | | | 1.5 | | | | | | | | | | | | |
| | | distearate*⁸ | | | | | | | | | | | | | | | | | | | | |
| | (F) | Limonene*⁹ | | | | | | | | | 0.3 | | | | | | | | | | | |
| | | Menthol*¹⁰ | | | | | | | | | | 0.5 | | | | | | | | | | |
| | (G) | Polypropylene glycol*¹¹ | | | | | | | | | | | 1.0 | | | | | | | | | |
| | | Benzyl alcohol*¹² | | | | | | | | | | | | 0.3 | | | | | | | | |
| | (H) | Riboflavin*¹³ | | | | | | | | | | | | | 0.1 | | | | | | | |
| | | D-panthenol*¹⁴ | | | | | | | | | | | | | | 0.1 | | | | | | |
| | (I) | Carboxyvinyl polymer*¹⁵ | | | | | | | | | | | | | | | 0.8 | | | | | |
| | | Hydroxyethyl cellulose*¹⁶ | | | | | | | | | | | | | | | | 1.0 | | | | |
| | (J) | Sodium chloride | | | | | | | | | | | | | | | | | 0.5 | | | |
| | | Sodium sulfate | | | | | | | | | | | | | | | | | | 2.0 | | |
| | (K) | EDTA | | | | | | | | | | | | | | | | | | | 0.5 | |
| | | HEDP | | | | | | | | | | | | | | | | | | | | 0.5 |

| | |
|---|---|
| pH adjuster | q.s. |
| Water | Balance |

*¹ to *¹⁶the same as those in Table 2

TABLE 4

| Cleansing | | Formula Examples | | | | | | | | | |
|---|---|---|---|---|---|---|---|---|---|---|---|
| composition | | 31 | 32 | 33 | 34 | 35 | 36 | 37 | 38 | 39 | 40 |
| (A) | Internal olefin sulfonate (1) | 8.0 | 8.0 | 8.0 | 8.0 | 9.0 | 7.0 | 8.0 | 8.0 | 4.0 | 12.0 |
| | Internal olefin sulfonate(2) | 2.0 | 2.0 | 2.0 | 2.0 | 1.0 | 3.0 | 2.0 | 2.0 | 1.0 | 3.0 |
| (B) | Sodium laurylphosphate*¹⁷ | | | | 3.0 | | | 6.0 | | | |
| | Sodium cocoyl glutamate*¹⁸ | | | 5.0 | | | | | | | |
| | Sodium cocoyl methyltaurine*¹⁹ | | | | | 2.0 | | | 5.0 | | |
| | Sodium laurylethersulfate*²⁰ | | | | 2.0 | | 5.0 | | 3.0 | 6.0 | |
| | Palm oil fatty acid amidepropylbetaine*²¹ | 1.5 | | | | 1.0 | | 1.0 | 1.0 | | |
| | Betaine lauryldimethylaminoacetate*²² | | 2.4 | | | | | | | | |
| | PPG-5CETETH-20*²³ | 0.5 | 0.5 | | | | | | | | |
| | Palm oil fatty acid monoethanolamide*²⁴ | | | 1.0 | 1.0 | | | | 1.0 | | 0.7 |
| | Cetyltrimethylammonium chloride*²⁵ | | | | | 0.1 | | | | | |
| (C) | Eucalyptus extract*²⁶ | 0.1 | | 0.1 | | | | | | | |
| | Chamomile flower extract*²⁷ | | | | 0.02 | | | 0.05 | | | |
| | Sugarcane extract | 0.1 | 0.05 | | | | | | | | |
| | Tea leaf extract | 0.1 | | | | | | | | | |
| | Lemone peel extract | 0.1 | | | | | | | | | |
| | Avocado extract | | 0.15 | | | | | | | | |
| | Sodium hyaluronate*²⁸ | | | | | 0.1 | | | 0.02 | | 0.05 |
| | Sodium pyrrolidonecarboxylate*²⁹ | | | | | 0.4 | | | | | |
| | Pseudo ceramide*³⁰ | | | 1.0 | | | | | | | |
| | Glycerol*³¹ | 1.2 | | | | | | | | | 4.0 |
| (D) | Triclocarban | | | | 0.2 | | | | 0.2 | | |
| | Piroctone olamine*³² | | | | | | 0.5 | | | | |
| | Zinc pyrithione | | | | | 0.5 | | | | | |
| | Benzalkonium cetyl phosphate*³³ | | | | | | | 1.0 | | | |
| | Salicylic acid | 0.1 | 0.5 | | | | 0.1 | | 0.2 | | |
| | Benzalkonium chloride*³⁴ | | | | 1.0 | | | | | | |
| (E) | Ethylene glycol distearate ³⁵ | | | 1.0 | | | | | | | |
| | Ethylene glycol distearate*³⁶ | | | | 1.0 | | | | | | |
| | Ethylene glycol distearate*³⁷ | | | | | 0.7 | | | | | |
| | Ethylene glycol distearate*³⁸ | | | | | | 1.4 | | | | |
| | Ethylene glycol distearate*³⁹ | | | | | | | | | | 1.0 |
| | Ethylene glycol distearate*⁴⁰ | | | | | | | | 1.0 | | |
| | Ethylene glycol distearate*⁴¹ | | | | | | | | | 1.0 | |
| | Mica | 0.2 | | | | | | | | 1.0 | |
| | Tin oxide | 0.2 | | | | | | | | 1.0 | |
| (F) | Linalool | | 0.2 | 0.05 | 0.03 | | 0.1 | | | 0.05 | |
| | Ambroxan | | | 0.05 | | | | | | | |
| | Eugenol | | | 0.05 | | | | | | | |
| | Pearlide | | | | 0.03 | | | | | | |
| | HELIOL | | | | 0.03 | | 0.05 | | | | |
| | Limonene*⁴² | 0.3 | 0.1 | 0.1 | | 0.3 | | 0.3 | | | 3.0 |
| | α-Isomethylionone | 0.1 | | | | | | | | | |
| | Hexylcinnamal | 0.1 | 0.1 | | | | | | | | |
| (G) | Benzyloxyethanol | | | 0.5 | 0.25 | | | | | | 0.1 |
| | Ethylene glycol dibutyl ether | | | | | 0.3 | | | | | |
| | Polyethylene glycol (average molecular | | | | | 0.3 | | | | | |
| | weight of 400) | | | | | | | | | | |
| | Polypropylene glycol*⁴³ | | | | 1.0 | | | | | | |
| (H) | Retinol | | | | | 0.05 | | | | | |
| | Dl-tocopherol | | | | | 0.05 | | | | | |
| | Ascorbic acid | | | | | 0.05 | | | 0.1 | | |
| | D-panthenol*⁴⁴ | | | | 0.1 | | | | | | |
| | Pyridoxine HCl (vitamin B)*⁴⁵ | 0.1 | 0.2 | | | | | | | | |
| (I) | Hydroxypropyl cellulose*⁴⁶ | | | | | 0.3 | | | | | |
| | Guar gum*⁴⁷ | | | | | | 0.2 | | | | |
| | Sodium polyacrylate*⁴⁸ | | | | | | | | | 0.3 | |
| | Carboxyvinyl polymer*⁴⁹ | | 0.5 | | 0.3 | | | 0.2 | | | 0.5 |
| | Acrylic acid alkyl copolymer*⁵⁰ | 0.3 | | | | | | | | | |
| (J) | Sodium chloride | | | 0.5 | | | 1.1 | | | | |
| | Trisodium citrate | | | | 0.2 | | | 0.4 | | | |
| | Disodium succinate | | | | | 0.35 | | | 0.3 | 0.3 | |
| (K) | EDTA-3Na*⁵¹ | | | 0.2 | | 0.05 | | 0.75 | | | |
| | Etidronic acid (diphosphonic acid)*⁵² | | | | 0.1 | | | | 0.02 | | |
| (L) | Silicone*⁵³ | 1.0 | | | 2.8 | | 1.4 | | | | |
| | Amino-modified silicone*⁵⁴ | | 0.5 | | | 0.3 | | | | | |
| | Behenyl alcohol*⁵⁵ | 0.1 | | | | | | | | | |
| | Glyceryl eicosanoate*⁵⁶ | 0.05 | 0.2 | | | | | | | | |
| (M) | Cationated guar gum*⁵⁷ | 0.3 | | | 0.2 | | 0.4 | | | | |
| | Diallyl quaternary ammonium salt polymer*⁵⁸ | | 0.2 | | | | | | 0.90 | | |
| | Diallyl quaternary ammonium salt/acrylamide | | | 0.2 | | | | | | | 0.1 |
| | copolymer*⁵⁹ | | | | | | | | | | |

| | |
|---|---|
| pH adjuster | q.s. |
| Water | Balance |

Physical property of the component (A) in Table 4: the content of C-2 position sulfonate group of 30% by mass, HAS/IOS = 80/20, the content of the inorganic compound of 1% by mass
*¹⁷made by Nikko Chemicals Co., Ltd., trade name: NIKKOL SLP-N
*¹⁸made by Ajinomoto Co., Inc., trade name: Amisoft CS-22 (effective component of 25%)
*¹⁹made by Nikko Chemicals Co., Ltd., trade name: NIKKOL CMT-30 (effective component of 30%)
*²⁰made by Kao Corporation, trade name: EMAL 270J (effective component of 70%)
*²¹made by Kao Corporation, trade name: AMPHITOL 55AB (effective component of 30%)
*²²made by Kao Corporation, trade name: AMPHITOL 20BS (effective component of 30%)
*²³made by Croda International Plc, trade name: Procetyl AWS
*²⁴made by Kawaken Fine Chemicals Co., Ltd., trade name: Amisol CME
*²⁵made by Kao Corporation, trade name: Cortamine 60W (effective component of 30%)
*²⁶made by ICHIMARU PHARCOS Co., Ltd., trade name: Pharcolex Eucalyptus B
*²⁷made by ICHIMARU PHARCOS Co., Ltd., trade name: Pharcolex BX44
*²⁸made by BASF Japan Ltd., trade name: Hyaluronic Filling Spheres
*²⁹made by Ajinomoto Co., Inc., trade name: PRODEW 300 (effective component of 10.55%)
*³⁰made by Kao Corporation, trade name: SOFCARE Ceramide SL-E
*³¹made by Kao Corporation, trade name: concentrated glycerol for cosmetics
*³²made by Clariant International Ltd., trade name: Octopirox
*³³made by Kao Corporation, trade name: SANISOL P (effective component of 50%)
*³⁴made by Kao Corporation, trade name: SANISOL C (effective component of 50%)
*³⁵made by Cognis GmbH, trade name: Euperlan PK1200 (effective component of 20%)
*³⁶made by Cognis GmbH, trade name: Euperlan PK771 (effective component of 23%)
*³⁷made by Cognis GmbH, trade name: Euperlan PK810 (effective component of 20%)
*³⁸made by Cognis GmbH, trade name: Euperlan PK900 (effective component of 25%)
*³⁹made by Kao Corporation, trade name: PEARL CONCENTRATE SA-M2 (effective component of 20%)
*⁴⁰made by Kao Corporation, trade name: PEARL CONCENTRATE FC-1 (effective component of 20%)
*⁴¹made by Kao Corporation, trade name: EMANON 3201M-V
*⁴²made by Yamakei Sangyo K.K.
*⁴³made by Sanyo Chemical Industries, Ltd., trade name: NEWPOL PP-1000
*⁴⁴made by BASF SE, trade name: D-panthenol 50P (effective component of 50%)
*⁴⁵made by DSM Nutritional Japan K.K., trade name: pyridoxine hydrochloride
*⁴⁶made by NIPPON SODA CO., LTD., trade name: CELNY M
*⁴⁷made by Evonik Goldschmidt GmbH, trade name: TEGO Emulprot
*⁴⁸made by ISP Japan K.K., trade name: LUBRAJEL IIXD FREE
*⁴⁹made by Lubrizol Advanced Materials, Inc., trade name: Carbopol 980
*⁵⁰made by Evonik Rohm GmbH, trade name: EUDRAGIT L100
*⁵¹made by Nagase ChemteX Corporation, trade name: Clewat 3Na
*⁵²made by Themphos International B.V., trade name: DEQUEST 2010CS (effective component of 60%)
*⁵³made by Dow Corning Toray Co., Ltd., trade name: BY22-050 (effective component of 50%)
*⁵⁴made by Dow Corning Toray Co., Ltd., trade name: BY22-079 (effective component of 14%)
*⁵⁵made by Kao Corporation, trade name: KALCOL 220-80
*⁵⁶made by The Nisshin OilliO Group, Ltd., trade name: Nomcort HK-G
*⁵⁷made by Rhodia S. A., trade name: Jaguar C-13S)
*⁵⁸made by The Lubrizol Corporation, trade name: MERQUART 550
*⁵⁹made by The Lubrizol Corporation, trade name: MERQUART 2001

TABLE 5

| | | | Formula Examples | | | | | | | | | |
|---|---|---|---|---|---|---|---|---|---|---|---|---|
| Cleansing composition | | | 41 | 42 | 43 | 44 | 45 | 46 | 47 | 48 | 49 | 50 |
| Blended | (A) | Internal olefin sulfonate (3) | 8.0 | 8.0 | 8.0 | 8.0 | 9.0 | 7.0 | 8.0 | 8.0 | 4.0 | 12.0 |
| composition | | Internal olefin sulfonate (4) | 2.0 | 2.0 | 2.0 | 2.0 | 1.0 | 3.0 | 2.0 | 2.0 | 1.0 | 3.0 |
| (parts by | (B) | Sodium laurylphosphate*¹⁷ | | | | 3.0 | | | 6.0 | | | |
| mass) | | Sodium cocoyl glutamate*¹⁸ | | | 5.0 | | | | | | | |
| | | Sodium cocoyl | | | | | 2.0 | | | 5.0 | | |
| | | methyltaurine*¹⁹ | | | | | | | | | | |
| | | Sodium laurylethersulfate*²⁰ | | | | 2.0 | | 5.0 | | 3.0 | 6.0 | |
| | | Palm oil fatty acid | 1.5 | | | | 1.0 | | 1.0 | 1.0 | | |
| | | amidepropylbetaine*²¹ | | | | | | | | | | |
| | | Betaine | | 2.4 | | | | | | | | |
| | | lauryldimethylaminoacetate*²² | | | | | | | | | | |
| | | PPG-5CETETH-20*²³ | 0.5 | 0.5 | | | | | | | | |
| | | Palm oil fatty acid | | | 1.0 | 1.0 | | | | 1.0 | | 0.7 |
| | | monoethanolamide*²⁴ | | | | | | | | | | |
| | | Cetyltrimethylammonium | | | | | 0.1 | | | | | |
| | | chloride*²⁵ | | | | | | | | | | |
| | (C) | Eucalyptus extract*²⁶ | 0.1 | | 0.1 | | | | | | | |
| | | Chamomile flower extract*²⁷ | | | | 0.02 | | | 0.05 | | | |
| | | Sugarcane extract | 0.1 | 0.05 | | | | | | | | |
| | | Tea leaf extract | 0.1 | | | | | | | | | |
| | | Lemone peel extract | 0.1 | | | | | | | | | |
| | | Avocado extract | | 0.15 | | | | | | | | |
| | | Sodium hyaluronate*²⁸ | | | | | 0.1 | | | 0.02 | | 0.05 |
| | | Sodium | | | | | 0.4 | | | | | |
| | | pyrrolidonecarboxylate*²⁹ | | | | | | | | | | |
| | | Pseudo ceramide*³⁰ | | | 1.0 | | | | | | | |
| | | Glycerol*³¹ | 1.2 | | | | | | | | | 4.0 |
| | (D) | Triclocarban | | | | 0.2 | | | | 0.2 | | |
| | | Piroctone olamine*³² | | | | | | 0.5 | | | | |
| | | Zinc pyrithione | | | | | 0.5 | | | | | |
| | | Benzalkonium cetyl | | | | | | | 1.0 | | | |
| | | phosphate*³³ | | | | | | | | | | |
| | | Salicylic acid | 0.1 | 0.5 | | | | 0.1 | | 0.2 | | |
| | | Benzalkonium chloride*³⁴ | | | | 1.0 | | | | | | |
| | (E) | Ethylene glycol distearate*³⁵ | | | 1.0 | | | | | | | |
| | | Ethylene glycol distearate*³⁶ | | | | 1.0 | | | | | | |
| | | Ethylene glycol distearate*³⁷ | | | | | 0.7 | | | | | |
| | | Ethylene glycol distearate*³⁸ | | | | | | 1.4 | | | | |
| | | Ethylene glycol distearate*³⁹ | | | | | | | | | | 1.0 |
| | | Ethylene glycol distearate*⁴⁰ | | | | | | | | 1.0 | | |
| | | Ethylene glycol distearate*⁴¹ | | | | | | | | | 1.0 | |
| | | Mica | 0.2 | | | | | | | | 1.0 | |
| | | Tin oxide | 0.2 | | | | | | | | 1.0 | |
| | (F) | Linalool | | 0.2 | 0.05 | 0.03 | | 0.1 | | | 0.05 | |
| | | Ambroxan | | | 0.05 | | | | | | | |
| | | Eugenol | | | 0.05 | | | | | | | |
| | | Pearlide | | | | 0.03 | | | | | | |
| | | HELIOL | | | | 0.03 | | 0.05 | | | | |
| | | Limonene*⁴² | 0.3 | 0.1 | 0.1 | | 0.3 | | 0.3 | | | 3.0 |
| | | α-Isomethylionone | 0.1 | | | | | | | | | |
| | | Hexylcinnamal | 0.1 | 0.1 | | | | | | | | |
| | (G) | Benzyloxyethanol | | | 0.5 | 0.25 | | | | | | 0.1 |
| | | Ethylene glycol dibutyl ether | | | | | 0.3 | | | | | |
| | | Polyethylene glycol (average | | | | | 0.3 | | | | | |
| | | molecular weight of 400) | | | | | | | | | | |
| | | Polypropylene glycol*⁴³ | | | | 1.0 | | | | | | |
| | (H) | Retinol | | | | | 0.05 | | | | | |
| | | Dl-tocopherol | | | | | 0.05 | | | | | |
| | | Ascorbic acid | | | | | 0.05 | | | 0.1 | | |
| | | D-panthenol*⁴⁴ | | | | 0.1 | | | | | | |
| | | Pyridoxine HCl (vitamin B)*⁴⁵ | 0.1 | 0.2 | | | | | | | | |
| | (I) | Hydroxypropyl cellulose*⁴⁶ | | | | | 0.3 | | | | | |
| | | Guar gum*⁴⁷ | | | | | | 0.2 | | | | |
| | | Sodium polyacrylate*⁴⁸ | | | | | | | | | 0.3 | |
| | | Carboxyvinyl polymer*⁴⁹ | | 0.5 | | 0.3 | | | 0.2 | | | 0.5 |
| | | Acrylic acid alkyl | 0.3 | | | | | | | | | |
| | | copolymer*⁵⁰ | | | | | | | | | | |
| | (J) | Sodium chloride | | | 0.5 | | | 1.1 | | | | |
| | | Trisodium citrate | | | | 0.2 | | | 0.4 | | | |
| | | Disodium succinate | | | | | 0.35 | | | 0.3 | 0.3 | |
| | (K) | EDTA-3Na*⁵¹ | | | 0.2 | | 0.05 | | 0.75 | | | |
| | | Etidronic acid (diphosphonic | | | | 0.1 | | | | 0.02 | | |
| | | acid)*⁵² | | | | | | | | | | |
| | (L) | Silicone*⁵³ | 1.0 | | | 2.8 | | 1.4 | | | | |
| | | Amino-modified silicone*⁵⁴ | | 0.5 | | | 0.3 | | | | | |
| | | Behenyl alcohol*⁵⁵ | 0.1 | | | | | | | | | |
| | | Glyceryl eicosanoate*⁵⁶ | 0.05 | 0.2 | | | | | | | | |
| | (M) | Cationated guar gum*⁵⁷ | 0.3 | | | 0.2 | | 0.4 | | | | |
| | | Diallyl quaternary ammonium | | 0.2 | | | | | | 0.90 | | |
| | | salt polymer*⁵⁸ | | | | | | | | | | |
| | | Diallyl quaternary ammonium | | | 0.2 | | | | | | | 0.1 |
| | | salt/acrylamide copolymer*⁵⁹ | | | | | | | | | | |

| | |
|---|---|
| pH adjuster | q.s. |
| Water | Balance |

*¹⁷ to *⁵⁹the same as those in Table 4

### (5) Hair Evaluation after Washing

Each of the following components was placed in a beaker and heated to 80° C., followed by mixing. After confirming homogeneous dissolution, the mixture was cooled to give a plain shampoo. A hair bundle (Japanese hair which was never subjected to treatment such as bleaching and hair coloring, approximately 20 cm long, 15 g) was washed with the plain shampoo thus obtained, whereby a tress for evaluation was obtained.

(Composition of the Plain Shampoo)

| (Component) | (% by mass) |
|---|---|
| Sodium polyoxyethylene lauryl ether sulfate | 11.3 |
| (42.0% as EMAL E-27C (the product of Kao Corporation, | |
| active content, 27% by mass)) | |
| Coconut oil fatty acid N-methyl ethanolamide | 3.0 |
| (AMINON C-11S (the product of Kao Corporation)) | |
| Citric acid | 0.2 |
| Methylparaben | 0.3 |
| Purified water | Balance |
| Total | 100.0 |

The tress for evaluation thus obtained was washed with each cleansing composition for hair, and was evaluated for the rinse feel after shampooing, combability of the hair after rinsing, softness, and manageability of the hair after drying, based on the following evaluation criteria and evaluation method.

(Evaluation Criteria and Evaluation Method)

Rinse Feel

5: Rinse feel is very good
4: Rinse feel is good
3: Rinse feel is ordinary (equal to that in Comparative Example 1)
2: Rinse feel is bad
1: Rinse feel is very bad

Finger Combability

5: Combability is very good
4: Combability is good
3: Combability is ordinary (equal to that in Comparative Example 2)

### 2: Combability is bad

1: Combability is very bad

Manageability

5: Manageability of hair is very good
4: Manageability of hair is good
3: Manageability of hair is ordinary (equal to that in Comparative Example 2)
2: Manageability of hair is bad
1: Manageability of hair is totally impossible

(6) Skin Evaluation after Washing

Five expert panelists washed their hands with each cleansing composition for skin, and evaluated the rinse feel after washing, rinsing, and the moist feeling to the skin after towel drying, based on the following evaluation criteria and evaluation method. It should be noted that the rinse feel was evaluated based on the same criteria as those used for hair.

Moist Feeling

5: Very moist

### 4: Moist

3: Fair (equivalent to Comparative Example 2)
2: Not moist
1: Not moist at all and feels roughness

### Example 51

### Hair Shampoo

A hair shampoo having the composition below was produced as follows. Purified water, methylparaben, and a surfactant were placed in a beaker, and heated to 80° C. while stirring these. It was checked that these were uniformly dissolved. Silicone was added to the mixture after the mixture was cooled to 60° C. or less. A fragrance was added to the mixture after the mixture was cooled to 45° C. or less. Then, the mixture was uniformly stirred. The mixture was cooled to room temperature. Then, the moisture content vaporized by heating was supplemented, and further stirring was performed for 30 minutes or more. The obtained hair shampoo was evaluated in accordance with the evaluation method.

| (Component) | (% by mass) |
|---|---|
| Sodium internal olefin sulfonate (3) | 10.0 |
| Sodium internal olefin sulfonate (4) | 2.5 |
| Polyoxyethylene (1) lauryletherammonium sulfate *1 | 2.0 |
| Lauric acid monoethanolamide | 0.8 |
| Ethylene glycol distearate *2 | 1.0 |
| Silicone *3 | 1.0 |
| Fragrance, methylparaben | q.s. |
| Purified water | Balance |
| Total | 100.0 |

*1: made by Kao Corporation, 0.29% of EMAL 170S-A (effective component of 70%) was added
*2: made by Cognis GmbH, 5% of Euperlan PK-810 (effective component of 20%) was added
*3: made by Dow Corning Toray Co., Ltd., 1.82% of silicone BY22-050A (effective component of 55%) was added

The hair shampoo had excellent rinse feel, good combability after rinsing and softness of hair, and feel of use having excellent manageability after drying.

### Example 52

### Facial Cleanser

A facial cleanser having the composition below was produced in the same manner as in Example 51, and was evaluated. The facial cleanser had excellent rinse feel, and feel of use having excellent moist feeling after towel dry.

| (Component) | (% by mass) |
|---|---|
| Sodium internal olefin sulfonate (4) | 15.0 |
| Palm oil fatty acid monoethanolamide | 2.0 |
| Highly polymerized dimethylsiloxane *1 | 3.0 |
| Cocoamido propylbetaine | 5.0 |
| pH adjuster | q.s. |
| Purified water | Balance |
| Total | 100.0 |

*1: made by Dow Corning Toray Co., Ltd., trade name; BY22-060 (effective component of 60%)

### INDUSTRIAL APPLICABILITY

The cleansing composition of the present invention can be favorably used in the fields of hair shampoo, body shampoo, facial cleanser, makeup remover, and hand soap, and the like, and further, it is also favorably applicable to animals such as dogs and cats.

What is claimed is:

1. A cleansing composition for skin or hair comprising an internal olefin sulfonate (A) having 12 or more and 24 or less carbon atoms.

2. The cleansing composition for skin or hair according to claim 1, wherein a content of an internal olefin sulfonate in which a sulfonate group is present at a C-2 position in the internal olefin sulfonate (A) is less than 20% by mass.

3. The cleansing composition for skin or hair according to claim 1, wherein a total content of an internal olefin sulfonate having 16 carbon atoms and an internal olefin sulfonate having 18 carbon atoms in the internal olefin sulfonate (A) is 50% by mass or more.

4. The cleansing composition for skin or hair according to claim 1, wherein a mass content ratio of the internal olefin sulfonate having 16 carbon atoms to the internal olefin sulfonate having 18 carbon atoms in the internal olefin sulfonate (A) (internal olefin sulfonate having 16 carbon atoms/internal olefin sulfonate having 18 carbon atoms) is from 50/50 to 99/1.

5. The cleansing composition for skin or hair according to claim 1, wherein a mass content ratio of a hydroxy form of the internal olefin sulfonate to an olefin form of the internal olefin sulfonate in the internal olefin sulfonate (A) (hydroxy form/olefin form) is from 50/50 to 100/0.

6. The cleansing composition for skin or hair according to claim 1, wherein the internal olefin sulfonate (A) is obtained by sulfonating an internal olefin having 12 or more and 24 or less carbon atoms, followed by neutralization and then hydrolysis.

7. The cleansing composition for skin or hair according to claim 1, further comprising a surfactant other than the internal olefin sulfonate (A).

8. The cleansing composition for skin or hair according to of claim 1, further comprising a moisturizing agent.

9. The cleansing composition for skin or hair according to claim 1, further comprising an antibacterial agent or an anti-dandruff agent.

10. The cleansing composition for skin or hair according to claim 1, further comprising a pearling agent.

11. The cleansing composition for skin or hair according to claim 1, further comprising a fragrance.

12. The cleansing composition for skin or hair according to claim 1, further comprising an organic solvent.

13. The cleansing composition for skin or hair according to claim 1, further comprising a vitamin agent.

14. The cleansing composition for skin or hair according to claim 1, further comprising a thickener.

15. The cleansing composition for skin or hair according to claim 1, further comprising a water-soluble salt.

16. The cleansing composition for skin or hair according to claim 1, further comprising a chelating agent.

17. The cleansing composition for skin or hair according to claim 1, further comprising an oil solution.

18. The cleansing composition for skin or hair according to claim 1, further comprising an amphoteric polymer or a cationic polymer.
